(19) United States
(12) Patent Application Publication
(10) Pub. No.: US 2025/0384533 A1
(43) Pub. Date: Dec. 18, 2025

(54) WEARABLE DEVICE FOR CORRECTING VISION DEFECT OF USER AND CONTROLLING METHOD THEREOF

(71) Applicant: Samsung Electronics Co., Ltd., Suwon-si (KR)

(72) Inventors: Sai Hemanth KASARANENI, Noida (IN); Vidhu SHARMA, Noida (IN); Iresha MAHESHWARI, Noida (IN)

(21) Appl. No.: 19/237,769

(22) Filed: Jun. 13, 2025

Related U.S. Application Data
(63) Continuation of application No. PCT/KR2025/008072, filed on Jun. 12, 2025.

(30) Foreign Application Priority Data
Jun. 14, 2024 (IN) 202411046174

(51) Int. Cl. G06T 5/73; A61B 3/036; A61H 5/00; G06F 3/01; G06T 5/60; G06T 19/00; G06V 10/62; G06V 10/764; G06V 10/82; G06V 40/18

(52) U.S. Cl. CPC G06T 5/73; A61H 5/00; G06F 3/013; G06F 3/017; G06T 5/60; G06T 19/006; G06V 10/62; G06V 10/764; G06V 10/82; G06V 40/193; G06V 40/197; A61B 3/036; A61H 2201/1604; A61H 2201/165; A61H 2201/5043; G06T 2207/20084; G06V 2201/03

(57) ABSTRACT

A method for controlling a wearable device configured to dynamically correct a vision defect of a user is provided. The method includes generating at least one first image frame for providing a virtual reality or an augmented reality through the wearable device, obtaining, through at least one camera of the wearable device, at least one second image frame of an ocular of the user, identifying at least one first feature from the at least one first image frame, wherein the at least one first feature comprises at least one of one or more content features or one or more display features, and one or more user gesture features, identifying at least one second feature from the at least one second image frame, wherein the at least one second feature comprises one or more ocular features of the user, based on the at least one first feature and the at least one second feature, determining a type of a refractive error of the user while the virtual reality or the augmented reality is provided, adjusting, based on the type of the refractive error, at least one of the identified content features or the identified display features, and based on the at least one of the adjusted content features or the adjusted display features, providing the virtual reality or the augmented reality.

### CROSS-REFERENCE TO RELATED APPLICATION(S)

This application is a continuation application, claiming priority under 35 U.S.C. § 365 (c), of an International application No. PCT/KR2025/008072, filed on Jun. 12, 2025, which is based on and claims the benefit of an Indian Complete patent application No. 202411046174, filed on Jun. 14, 2024, in the Indian Patent Office, the disclosure of which is incorporated by reference herein in its entirety.

### FIELD OF THE INVENTION

The disclosure relates to the field of computer vision. More particularly, the disclosure relates to a wearable device for correcting vision defect of user and controlling method thereof.

### BACKGROUND

Computer vision is a field of artificial intelligence (AI) and virtual reality (VR) that deals with enabling computers and systems to derive meaningful information from digital images, videos, and other visual inputs. The computer vision may include one or more functionalities such as image acquisition, image processing, etc. The image processing involves techniques to enhance, filter, segment, and extract meaningful information from visual data.

In the context of augmented reality (AR) and VR entertainment, computer vision techniques play a pivotal role in an integration of digital content with the user's physical or virtual environment. This integration is particularly evident in a video see through head mounted display (VST-HMD) device. The VST-HMD device may employ camera sensors to capture a user's real-world view, and then utilize advanced computer vision algorithms to overlay or blend virtual elements, such as three-dimensional (3D) models, text, and graphics, with a captured video feed. This process enables the user to perceive a seamless augmented reality experience, where the virtual and physical elements are harmoniously integrated. By leveraging these computer vision capabilities, the VST-HMD devices can enhance the user's perception and interaction with the combined virtual and physical worlds, paving a way for more advanced and engaging AR and VR applications.

The above information is presented as background information only to assist with an understanding of the disclosure. No determination has been made, and no assertion is made, as to whether any of the above might be applicable as prior art with regard to the disclosure.

### SUMMARY

However, several problems are encountered in the existing VST-HMD device, in scenarios where the users have vision/eyesight defects 101 (e.g., myopia, hypermetropia, or astigmatism), as illustrated in FIG. 1, the viewing experience is hindered due to the user's low perceiving power, which are mentioned below.

FIG. 1 illustrates one or more problems associated with existing VST-HMD devices, according to the related art.

In the case of a user with a vision defect (refractive error), if the user uses the existing VST-HMD device without their corrective glasses 102, as illustrated in FIG. 1, the projected content may appear out of focus unless the focus is manually adjusted. To nullify the vision defect (refractive error) certain existing systems may provide one or more solutions. One solution, the user has to wear the existing VST-HMD device over their glasses (e.g., Oculus Rift device and HTC Vive device, etc.). However, this arrangement can lead to an uncomfortable situation for the user, as it puts additional pressure on the nose and ears supporting both the glasses and the existing VST-HMD device, which is undesirable. Another solution, the user has to purchase an extra piece of hardware, such as prescription lenses, to be used with the VST-HMD device (e.g., VR headsets like the Apple Vision Pro provide extra lenses based on the user's eyesight), which can be magnetically attached to the headset. However, this solution increases the overall cost of the already expensive hardware 103, as illustrated in FIG. 1.

Thus, it is desired to address the above-mentioned disadvantages or other shortcomings or at least provide a useful alternative for dynamically correcting the vision defect of the user while wearing the VST-HMD device.

Aspects of the disclosure are to address at least the above-mentioned problems and/or disadvantages and to provide at least the advantages described below. Accordingly, an aspect of the disclosure is to provide a wearable device (e.g., Video See Through Head Mounted Display (VST-HMD) device) for correcting vision defect of user and controlling method thereof.

Additional aspects will be set forth in part in the description which follows and, in part, will be apparent from the description, or may be learned by practice of the presented embodiments.

In accordance with an aspect of the disclosure, a method for dynamically correcting a vision defect of a user using a video see through head mounted display (VST-HMD) device is provided. The method includes generating a plurality of first image frames and a plurality of second image frames through one or more image sensors of the VST-HMD device, extracting a plurality of first feature sets from the plurality of generated first image frames, wherein the plurality of first feature sets includes at least one of one or more content features, one or more display features, and one or more user gesture features, extracting a plurality of second feature sets from the plurality of generated second image frames, wherein the plurality of second feature sets includes one or more ocular features, determining a refractive error experienced by the user while watching the plurality of generated first image frames through the VST-HMD device, and adjusting, based on the determined refractive error, the one or more extracted content features and the one or more extracted display features for dynamically correcting the vision defect of the user.

In accordance with another aspect of the disclosure, a system for dynamically correcting a vision defect of a user using a video see through head mounted display (VST-HMD) device is provided. The system includes memory storing one or more computer programs, a communicator, an intelligent vision enhancement module, and one or more processors communicatively coupled to the memory, the communicator, and the intelligent vision enhancement module, wherein the one or more computer programs include computer-executable instructions that, when executed by the one or more processors individually or collectively, cause the system to generate a plurality of first image frames and a plurality of second image frames through one or more image sensors of the VST-HMD device, extract a plurality of first feature sets from the plurality of generated first image frames, wherein the plurality of first feature sets includes at least one of one or more content features, one or more display features, and one or more user gesture features, extract a plurality of second feature sets from the plurality of generated second image frames, wherein the plurality of second feature sets includes one or more ocular features, determine a refractive error experienced by the user while watching the plurality of generated first image frames through the VST-HMD device, and adjust, based on the determined refractive error, the one or more extracted content features and the one or more extracted display features for dynamically correcting the vision defect of the user.

In accordance with another aspect of the disclosure, one or more non-transitory computer-readable storage media storing one or more computer programs including computer-executable instructions that, when executed by one or more processors of an electronic device individually or collectively, cause the electronic device to perform operations are provided. The operations include generating a plurality of first image frames and a plurality of second image frames through one or more image sensors of the VST-HMD device, extracting a plurality of first feature sets from the plurality of generated first image frames, wherein the plurality of first feature sets comprising at least one of one or more content features, one or more display features, and one or more user gesture features, extracting a plurality of second feature sets from the plurality of generated second image frames, wherein the plurality of second feature sets comprising one or more ocular features, determining a refractive error experienced by the user while watching the plurality of generated first image frames through the VST-HMD device, and adjusting, based on the determined refractive error, the one or more extracted content features and the one or more extracted display features for dynamically correcting a vision defect of the user.

In accordance with another aspect of the disclosure, a method for controlling a wearable device provided. The method comprises generating at least one first image frame for providing a virtual reality or an augmented reality through the wearable device, obtaining, through at least one camera of the wearable device, at least one second image frame of an ocular of the user, identifying at least one first feature from the at least one first image frame, wherein the at least one first feature comprises at least one of one or more content features or one or more display features, and one or more user gesture features, identifying at least one second feature from the at least one second image frame, wherein the at least one second feature comprises one or more ocular features of the user, based on the at least one first feature and the at least one second feature, determining a type of a refractive error of the user while the virtual reality or the augmented reality is provided, adjusting, based on the type of the refractive error, at least one of the identified content features or the identified display features, and based on the at least one of the adjusted content features or the adjusted display features, providing the virtual reality or the augmented reality.

In accordance with another aspect of the disclosure, a wearable device is provided. The wearable device includes at least one camera, memory storing one or more computer programs, one or more processors communicatively coupled to the at least one camera and the memory, wherein the one or more computer programs include computer-executable instructions that, when executed by the one or more processors individually or collectively, cause the wearable device to, generate at least one first image frame for providing a virtual reality or an augmented reality through the wearable device, obtain, through the at least one camera, at least one second image frame of an ocular of the user, identify at least one first feature from the at least one first image frame, wherein the at least one first feature comprises at least one of one or more content features or one or more display features, and one or more user gesture features, identify at least one second feature from the at least one second image frame, wherein the at least one second feature comprises one or more ocular features of the user, based on the at least one first feature and the at least one second feature, determine a type of a refractive error of the user while the virtual reality or the augmented reality is provided, adjust, based on the type of the refractive error, at least one of the identified content features or the identified display features, and based on the at least one of the adjusted content features or the adjusted display features, providing the virtual reality or the augmented reality.

In accordance with another aspect of the disclosure, one or more non-transitory computer-readable storage media are provided. The one or more non-transitory computer-readable storage media are configured to store one or more computer programs including computer-executable instructions that, when executed by one or more processors of a wearable device individually or collectively, cause the wearable device to perform operations, the operations comprising, generating at least one first image frame for providing a virtual reality or an augmented reality through the wearable device, obtaining, through at least one camera of the wearable device, at least one second image frame of an ocular of the user, identifying at least one first feature from the at least one first image frame, wherein the at least one first feature comprises at least one of one or more content features or one or more display features, and one or more user gesture features, identifying at least one second feature from the at least one second image frame, wherein the at least one second feature comprises one or more ocular features of the user, based on the at least one first feature and the at least one second feature, determining a type of a refractive error of the user while the virtual reality or the augmented reality is provided, adjusting, based on the type of the refractive error, at least one of the identified content features or the identified display features, and based on the at least one of the adjusted content features or the adjusted display features, providing the virtual reality or the augmented reality.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 illustrates one or more problems associated with existing video see-through head mounted display (VST-HMD) devices, according to the related art;

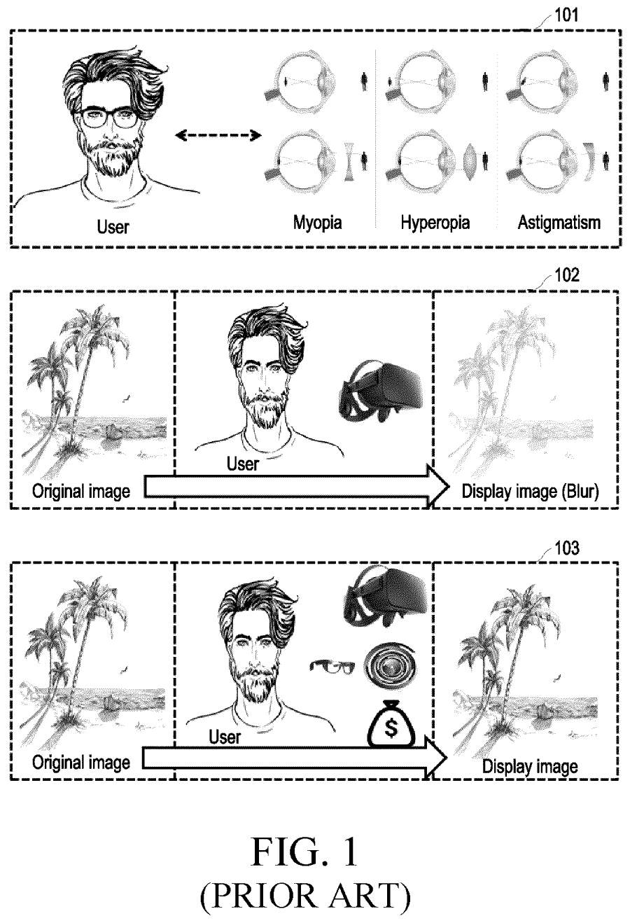

FIG. 2 illustrates a block diagram of a wearable device (e.g., VST-HMD device) for dynamically correcting a vision defect of a user, according to an embodiment of the disclosure;

FIG. 3 is a system flow diagram illustrating the method for dynamically correcting the vision defect of the user using the VST-HMD device, according to an embodiment of the disclosure;

FIG. 4 illustrates a scenario where a content hue detection unit and a content contrast detection unit of the VST-HMD device execute one or more operations to extract one or more content features, according to an embodiment of the disclosure;

FIG. 5 illustrates a scenario where a content depth detection unit of the VST-HMD device executes one or more operations to extract the one or more content features, according to an embodiment of the disclosure;

FIG. 6 illustrates a scenario where a spatial focus demand estimating unit of the VST-HMD device executes one or more operations to extract the one or more content features, according to an embodiment of the disclosure;

FIG. 7 illustrates a scenario where a user gesture recognizer of the VST-HMD device executes one or more operations to extract the one or more user gesture features, according to an embodiment of the disclosure;

FIG. 8 is a flow diagram illustrating a method for determining one or more ocular features by a pupil size determining unit of the VST-HMD device, according to an embodiment of the disclosure;

FIG. 9 illustrates a scenario where a spatial focus time tracking unit of the VST-HMD device executes one or more operations to extract the one or more ocular features, according to an embodiment of the disclosure;

FIG. 10 illustrates a scenario where an eye blinking rate calculating unit of the VST-HMD device executes one or more operations to extract the one or more ocular features, according to an embodiment of the disclosure;

FIG. 11 illustrates a scenario where an eye-opening ratio determining unit of the VST-HMD device executes one or more operations to extract the one or more ocular features, according to an embodiment of the disclosure;

FIG. 12 illustrates a scenario where an eye's tear condition detection unit of the VST-HMD device executes one or more operations to extract the one or more ocular features, according to an embodiment of the disclosure;

FIG. 13 illustrates a scenario where a refractive error generator of the VST-HMD device executes one or more operations to determine a refractive error experienced by the user while watching a plurality of generated first image frames through the VST-HMD device, according to an embodiment of the disclosure;

FIG. 14 illustrates a scenario where a refractive risk score generator of the VST-HMD device executes one or more operations to determine a refractive risk score of the user, according to an embodiment of the disclosure;

FIG. 15 illustrates a scenario where a target feature detector of the VST-HMD device executes one or more operations to determine a target features variation vector, according to an embodiment of the disclosure;

FIG. 16 illustrates one or more scenarios associated with a virtual exercise adviser of the VST-HMD device, according to an embodiment of the disclosure;

FIG. 17 illustrates a scenario where a feature modifier of the VST-HMD device executes one or more operations to adjust one or more extracted content features and one or more extracted display features for dynamically correcting the vision defect of the user, according to an embodiment of the disclosure;

FIG. 18 is a flow diagram illustrating a method for dynamically correcting a vision defect of the user using the VST-HMD device, according to an embodiment of the disclosure; and

FIGS. 19A and 19B are diagrams illustrating a wearable device according to various embodiments of the disclosure.

The same reference numerals are used to represent the same elements throughout the drawings.

### DETAILED DESCRIPTION OF FIGURES

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

Reference throughout this specification to “an aspect,” “another aspect” or similar language means that a particular feature, structure, or characteristic described in connection with the embodiment is included in at least one embodiment of the disclosure. Thus, appearances of the phrase “in an embodiment,” “in one embodiment,” “in another embodiment,” and similar language throughout this specification may, but do not necessarily, all refer to the same embodiment.

The terms “comprise,” “comprising,” or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Also, the various embodiments described herein are not necessarily mutually exclusive, as some embodiments can be combined with one or more other embodiments to form new embodiments. The term “or” as used herein, refers to a non-exclusive or unless otherwise indicated. The examples used herein are intended merely to facilitate an understanding of ways in which the embodiments herein can be practiced and to further enable those skilled in the art to practice the embodiments herein. Accordingly, the examples should not be construed as limiting the scope of the embodiments herein.

As is traditional in the field, embodiments may be described and illustrated in terms of blocks that carry out a described function or functions. These blocks, which may be referred to herein as units or modules or the like, are physically implemented by analog or digital circuits such as logic gates, integrated circuits, microprocessors, microcontrollers, memory circuits, passive electronic components, active electronic components, optical components, hardwired circuits, or the like, and may optionally be driven by firmware and software. The circuits may, for example, be embodied in one or more semiconductor chips, or on substrate supports such as printed circuit boards and the like. The circuits constituting a block may be implemented by dedicated hardware, or by a processor (e.g., one or more programmed microprocessors and associated circuitry), or by a combination of dedicated hardware to perform some functions of the block and a processor to perform other functions of the block. Each block of the embodiments may be physically separated into two or more interacting and discrete blocks without departing from the scope of the disclosure. Likewise, the blocks of the embodiments may be physically combined into more complex blocks without departing from the scope of the disclosure.

As such, the disclosure should be construed to extend to any alterations, equivalents, and substitutes in addition to those which are particularly set out in the accompanying drawings. Although the terms first, second, etc. may be used herein to describe various elements, these elements should not be limited by these terms. These terms are generally only used to distinguish one element from another.

It should be appreciated that the blocks in each flowchart and combinations of the flowcharts may be performed by one or more computer programs which include instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g. a central processing unit (CPU)), a communication processor (CP, e.g., a modem), a graphics processing unit (GPU), a neural processing unit (NPU) (e.g., an artificial intelligence (AI) chip), a wireless fidelity (Wi-Fi) chip, a Bluetooth® chip, a global positioning system (GPS) chip, a near field communication (NFC) chip, connectivity chips, a sensor controller, a touch controller, a finger-print sensor controller, a display driver integrated circuit (IC), an audio CODEC chip, a universal serial bus (USB) controller, a camera controller, an image processing IC, a microprocessor unit (MPU), a system on chip (SoC), an IC, or the like.

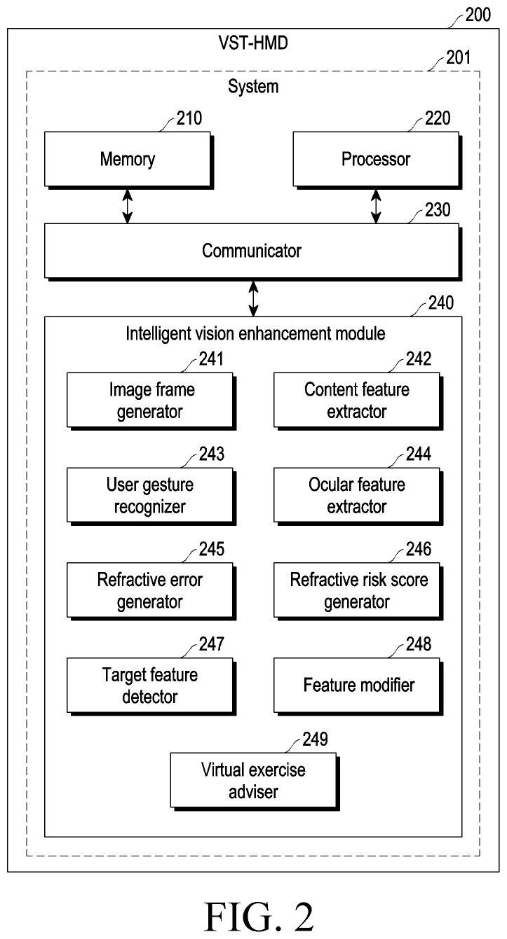

Referring now to the drawings, and more particularly to FIGS. 2 to 18, where similar reference characters denote corresponding features consistently throughout the figures, there are shown preferred embodiments.

FIG. 2 illustrates a block diagram of a VST-HMD device for dynamically correcting a vision defect of a user, according to an embodiment of the disclosure.

Referring to FIG. 2, examples of a VST-HMD device 200 may include, but are not limited to a Samsung gear, etc.

In an embodiment, the VST-HMD device 200 comprises a system 201. The system 201 may include memory 210, a processor 220, a communicator 230, and an intelligent vision enhancement module 240. In one or more embodiments, the system 201 may be implemented on one or multiple electronic devices (not shown in the FIG).

In an embodiment, the memory 210 stores instructions to be executed by the processor 220 for dynamically correcting the vision defect of the user using the VST-HMD device 200, as discussed throughout the disclosure. The memory 210 may include non-volatile storage elements. Examples of such non-volatile storage elements may include magnetic hard discs, optical discs, floppy discs, flash memories, or forms of electrically programmable read only memories (EPROM) or electrically erasable and programmable ROM (EEPROM) memories. In addition, the memory 210 may, in some examples, be considered a non-transitory storage medium. The term “non-transitory” may indicate that the storage medium is not embodied in a carrier wave or a propagated signal. However, the term “non-transitory” should not be interpreted that the memory 210 is non-movable. In some examples, the memory 210 can be configured to store larger amounts of information than the memory. In certain examples, a non-transitory storage medium may store data that can, over time, change (e.g., in random access memory (RAM) or cache). The memory 210 can be an internal storage unit, or it can be an external storage unit of the VST-HMD device 200, a cloud storage, or any other type of external storage.

The processor 220 communicates with the memory 210, the communicator 230, and the intelligent vision enhancement module 240. The processor 220 is configured to execute instructions stored in the memory 210 and to perform various for dynamically correcting the vision defect of the user using the VST-HMD device 200, as discussed throughout the disclosure. The processor 220 may include one or a plurality of processors, may be a general-purpose processor, such as a central processing unit (CPU), an application processor (AP), or the like, a graphics-only processing unit such as a graphics processing unit (GPU), a visual processing unit (VPU), and/or an artificial intelligence (AI) dedicated processor such as a neural processing unit (NPU).

The communicator 230 is configured for communicating internally between internal hardware components and with external devices (e.g., server) via one or more networks (e.g., radio technology). The communicator 230 includes an electronic circuit specific to a standard that enables wired or wireless communication.

In one embodiment, the system 201 may include a display module (not shown in the FIG.). The display module may accept user inputs and is made of a liquid crystal display (LCD), a light emitting diode (LED), an organic light emitting diode (OLED), or another type of display. The user inputs may include, but are not limited to, touch, swipe, drag, gesture, and so on.

In one embodiment, the system 201 may include a camera module (not shown in the FIG. 2). The camera module may include one or more image sensors (e.g., charged coupled device (CCD), complementary metal-oxide semiconductor (CMOS)) to capture one or more images/image frames/video to be processed for dynamically correcting the vision defect of the user using the VST-HMD device 200. In an alternative embodiment, the camera module may not be present, and the system 201 may process an image/video received from an external device or process a pre-stored image/video displayed at the display module.

In one or more embodiments, the intelligent vision enhancement module 240 may include an image frame generator 241, a content feature extractor 242, a user gesture recognizer 243, an ocular feature extractor 244, a refractive error generator 245, a refractive risk score generator 246, a target feature detector 247, a feature modifier 248, and a virtual exercise adviser 249. The intelligent vision enhancement module 240 is implemented by processing circuitry such as logic gates, integrated circuits, microprocessors, microcontrollers, memory circuits, passive electronic components, active electronic components, optical components, hardwired circuits, or the like, and may optionally be driven by firmware. The circuits may, for example, be embodied in one or more semiconductor chips, or on substrate supports such as printed circuit boards and the like.

In one or more embodiments, the image frame generator 241 is configured to generate a plurality of first image frames and a plurality of second image frames through one or more image sensors of the VST-HMD device 200. A first image frame of the plurality of first image frames may represent the display image that is shown on a screen of the VST-HMD device 200. The first image frame may be a virtual or augmented reality scene that is rendered and displayed for the user. The first image frame may also include an original image frame captured by the camera module of the VST-HMD device 200, which shows a real-world environment that the user is viewing through a transparent screen. A second image frame of the plurality of second image frames is an ocular image frame, which captures the user's eyes and eye movements. The ocular image frame may be used to determine the user's gaze direction and focus within a displayed content.

For instance, a user puts on the VST-HMD device 200 to test the latest AR application being developed for the device. As the user looks around a room, the image frame generator 241 captures the plurality of first image frames, which may include display image frames and original image frames. The display image frames are the virtual or augmented reality scenes that are rendered and displayed on the transparent screen of the VST-HMD device 200. The user sees a virtual 3D model of a new product design overlaid on the real-world environment. The original image frames are the camera-captured frames of an actual physical environment that the user is viewing through the transparent screen of the VST-HMD device 200. The user can see the real-world objects and people in the room, blended with virtual content.

Simultaneously, the image frame generator 241 also captures the plurality of second image frames, which are the ocular image frames. These ocular image frames record the user's eye movements and gaze patterns as she interacts with the AR application. The user can then use the data from the first and second image frames to fine-tune the AR application, ensuring a seamless and immersive experience for the end-users. For example, the eye-tracking data from the second image frames can be used to implement foveated rendering, where a display resolution is increased in the areas of the user's focus to enhance visual clarity.

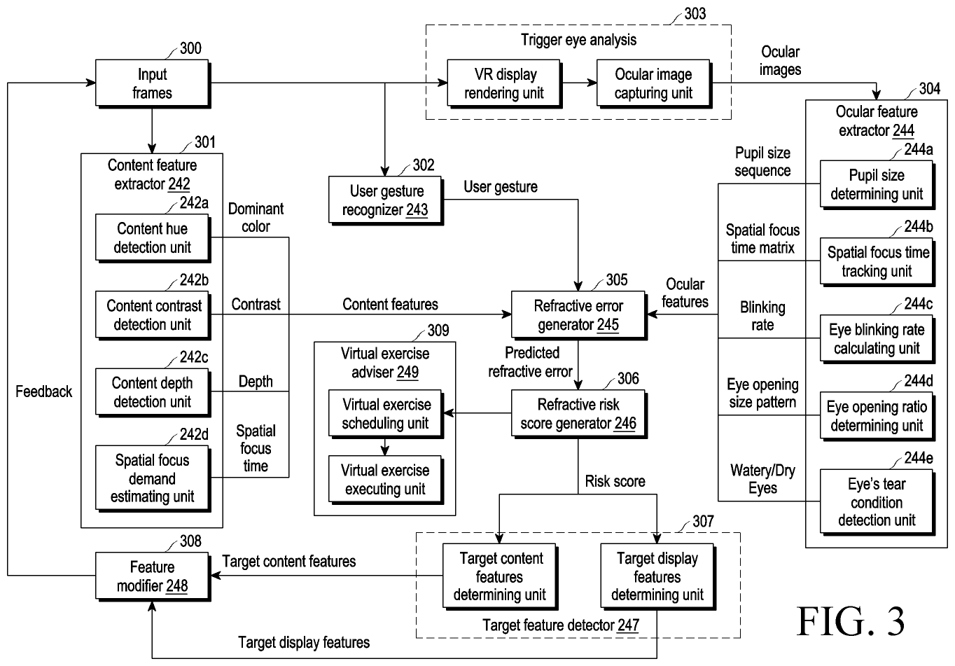

In one or more embodiments, the content feature extractor 242 is configured to extract a plurality of first feature sets from the plurality of generated first image frames, as described in conjunction with FIGS. 3 to 6. The plurality of first feature sets may include, but are not limited to, one or more content features and one or more display features. The one or more content features represent a characteristic of a visual content depicted in the plurality of generated first image frames may include, but are not limited to, object recognition information, scene classification information, text identification information, high-level semantic information, dominant color information, contrast information, depth information, and spatial focus time information. The one or more display features represent a characteristic of a visual presentation and layout of the visual content depicted in the plurality of generated first image frames may include, but are not limited to, a screen size, a screen resolution, an aspect ratio, a color profile, a brightness of the screen, a wavelength, or other display-specific properties.

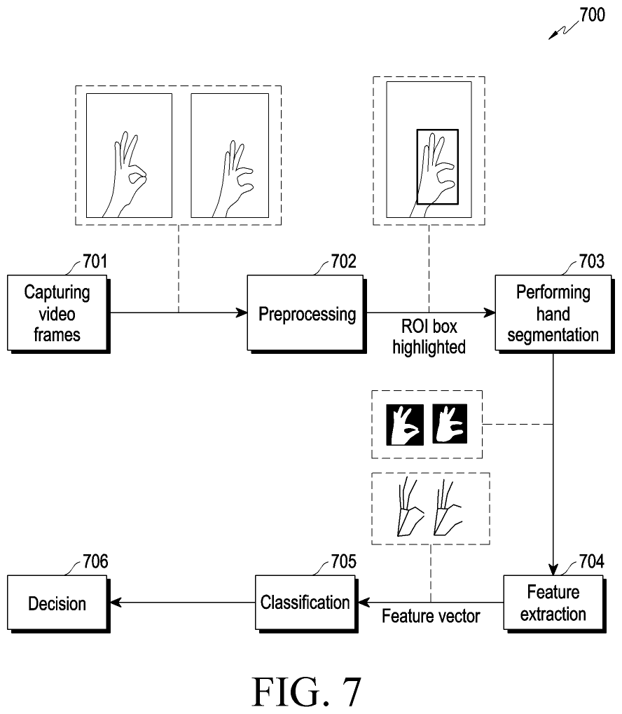

In one or more embodiments, the user gesture recognizer 243 is configured to extract the plurality of first feature sets from the plurality of generated first image frames, as described in conjunction with FIG. 7. The plurality of first feature sets may include one or more user gesture features. The one or more user gesture features represent a characteristic of one or more motions and movements captured in the plurality of generated first image frames may include, but are not limited to, one or more hand gestures, one or more body postures, or other physical interactions with visual content.

In one or more embodiments, the ocular feature extractor 244 is configured to extract a plurality of second feature sets from the plurality of generated second image frames, as described in conjunction with FIGS. 3 and 8 to 12. The plurality of second feature sets comprising one or more ocular features. The one or more ocular features represent a characteristic of one or more eye motions and eye movements of the user of the VST-HMD device 200 captured in the plurality of generated second image frames may include, but are not limited to, pupil size information, spatial focus time matrix information, blinking rate information, eye-opening size pattern information, and eye tear condition information.

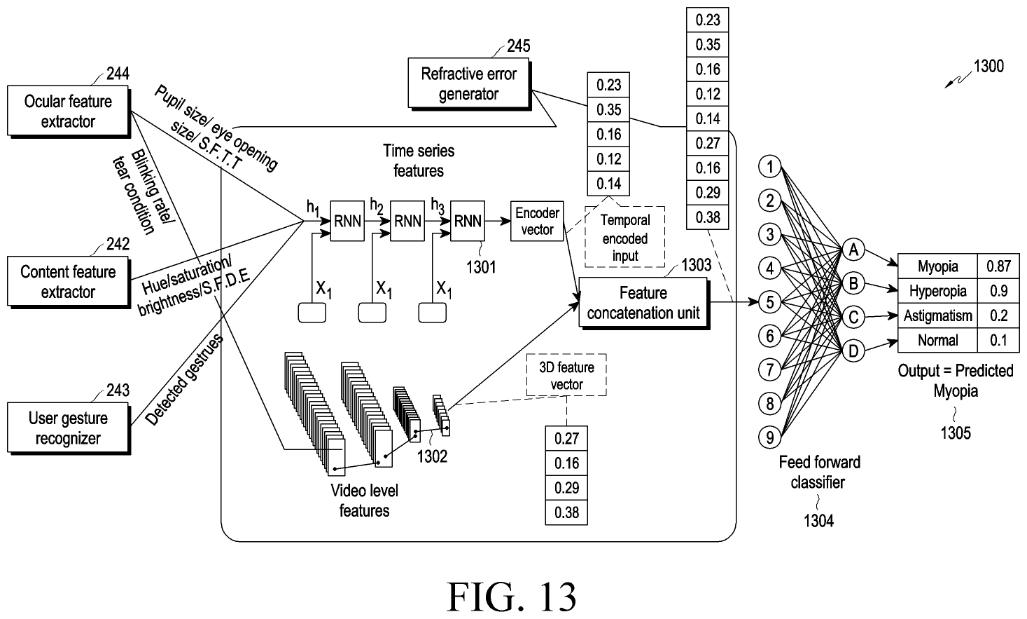

In one or more embodiments, the refractive error generator 245 is configured to determine a refractive error experienced by the user while watching the plurality of generated first image frames through the VST-HMD device 200, as described in conjunction with FIGS. 3 and 13. The refractive error is determined based on the plurality of extracted first feature sets and the plurality of extracted second feature sets.

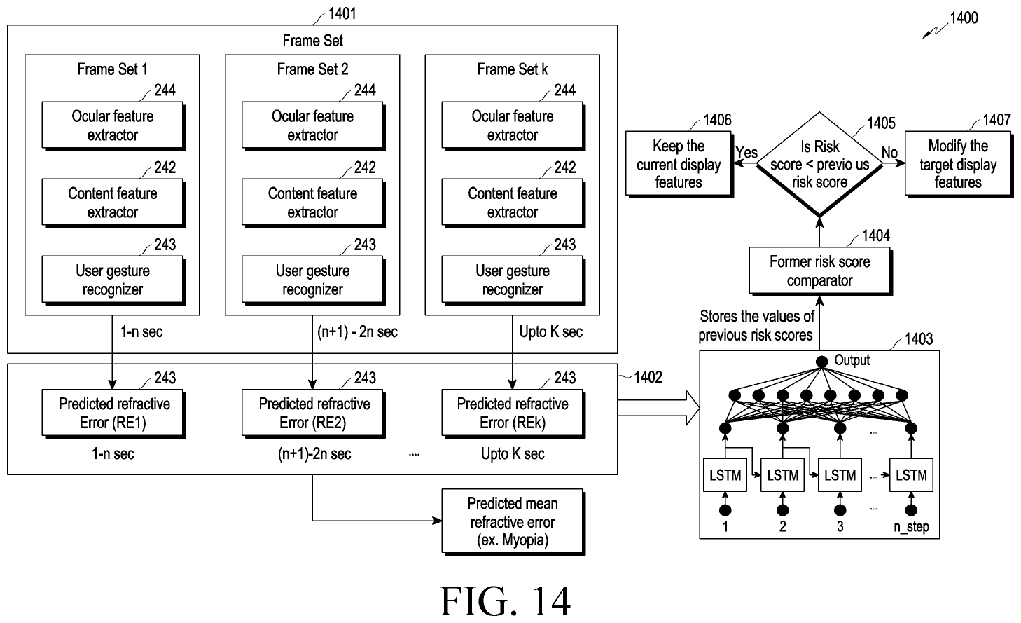

In one or more embodiments, the refractive risk score generator 246 is configured to determine a refractive risk score of the user, as described in conjunction with FIGS. 3 and 14. The refractive risk score of the user is determined based on the plurality of extracted first feature sets, the plurality of extracted second feature sets, and the determined refractive error.

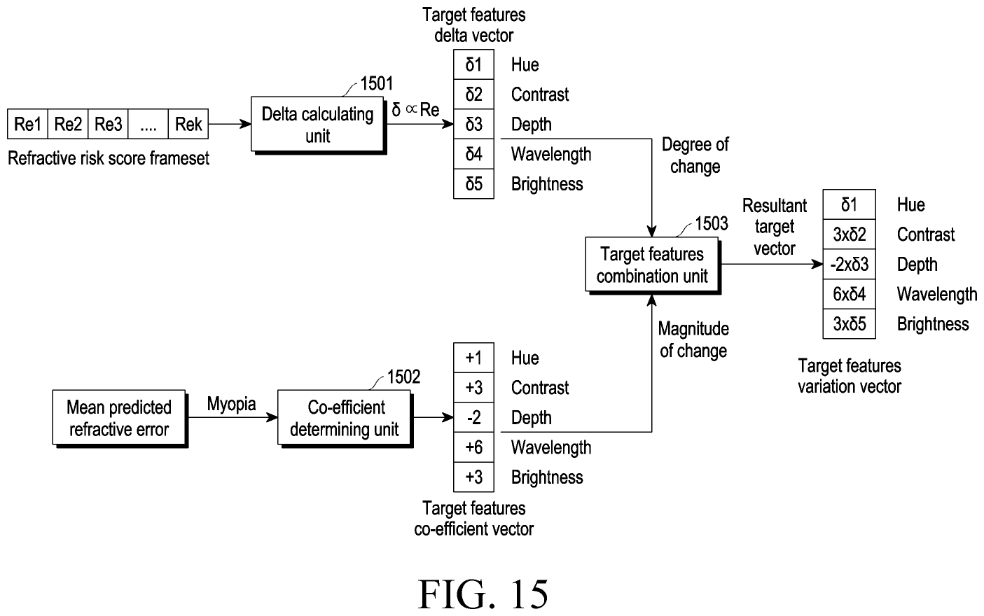

In one or more embodiments, the target feature detector 247 is configured to determine a target feature variation vector based on the refractive risk score and a mean predicted refractive error, as described in conjunction with FIGS. 3 and 15.

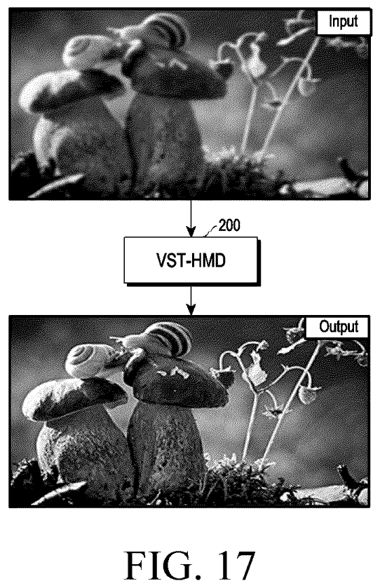

In one or more embodiments, the feature modifier 248 is configured to adjust, based on the determined refractive error, the one or more extracted content features and the one or more extracted display features for dynamically correcting the vision defect of the user, as described in conjunction with FIGS. 3 and 17. In one embodiment, the feature modifier 248 is configured to adjust the one or more extracted content features and the one or more extracted display features, in order to mitigate the determined refractive risk score, for dynamically correcting the vision defect of the user.

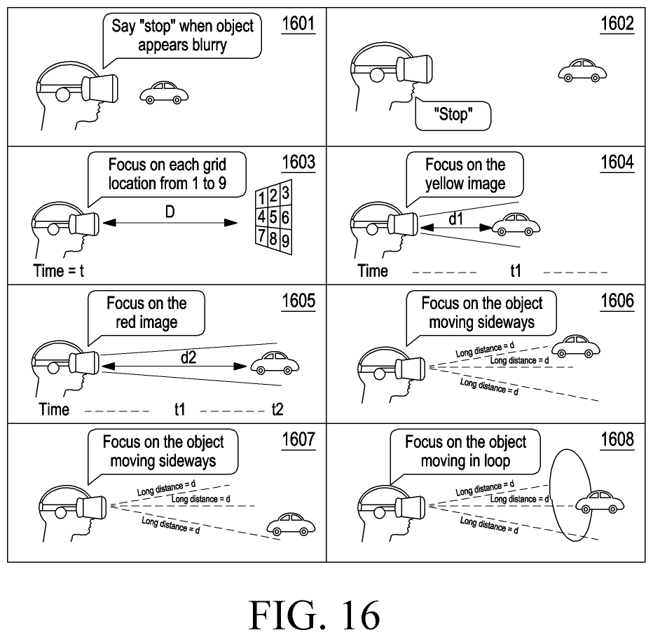

In one or more embodiments, the virtual exercise adviser 249 is configured to recommend one or more personalized virtual training exercises for prolonged improvement of the vision defect of the user, as described in conjunction with FIGS. 3 and 16.

A function associated with the various components of the intelligent vision enhancement module 240 may be performed through the non-volatile memory, the volatile memory, and the processor 220. One or a plurality of processors controls the processing of the input data in accordance with a predefined operating rule or AI model stored in the non-volatile memory and the volatile memory. The predefined operating rule or AI model is provided through training or learning to dynamically correct the vision defect of the user. Here, being provided through learning means that, by applying a learning algorithm to a plurality of learning data, a predefined operating rule or AI model of the desired characteristic is made. The learning may be performed in a device itself in which AI according to an embodiment is performed, and/or may be implemented through a separate server/system. The learning algorithm is a method for training a predetermined target device (for example, a robot) using a plurality of learning data to cause, allow, or control the target device to decide or predict. Examples of learning algorithms include, but are not limited to, supervised learning, unsupervised learning, semi-supervised learning, or reinforcement learning.

The AI model may consist of a plurality of neural network layers. Each layer has a plurality of weight values and performs a layer operation through a calculation of a previous layer and an operation of a plurality of weights. Examples of neural networks include, but are not limited to, convolutional neural network (CNN), deep neural network (DNN), recurrent neural network (RNN), restricted Boltzmann machine (RBM), deep belief network (DBN), bidirectional recurrent deep neural network (BRDNN), generative adversarial networks (GAN), and deep Q-networks.

Although FIG. 2 shows various hardware components of the VST-HMD device 200, but it is to be understood that other embodiments are not limited thereon. In other embodiments, the VST-HMD device 200 may include less or more number of components. Further, the labels or names of the components are used only for illustrative purposes and do not limit the scope of the disclosure. One or more components can be combined to perform the same or substantially similar functions to dynamically correct the vision defect of the user.

FIG. 3 is a system flow diagram illustrating the method for dynamically correcting the vision defect of the user using the VST-HMD device, according to an embodiment of the disclosure.

The method may execute multiple operations to dynamically correct the vision defect of the user using the VST-HMD device 200, which are given below.

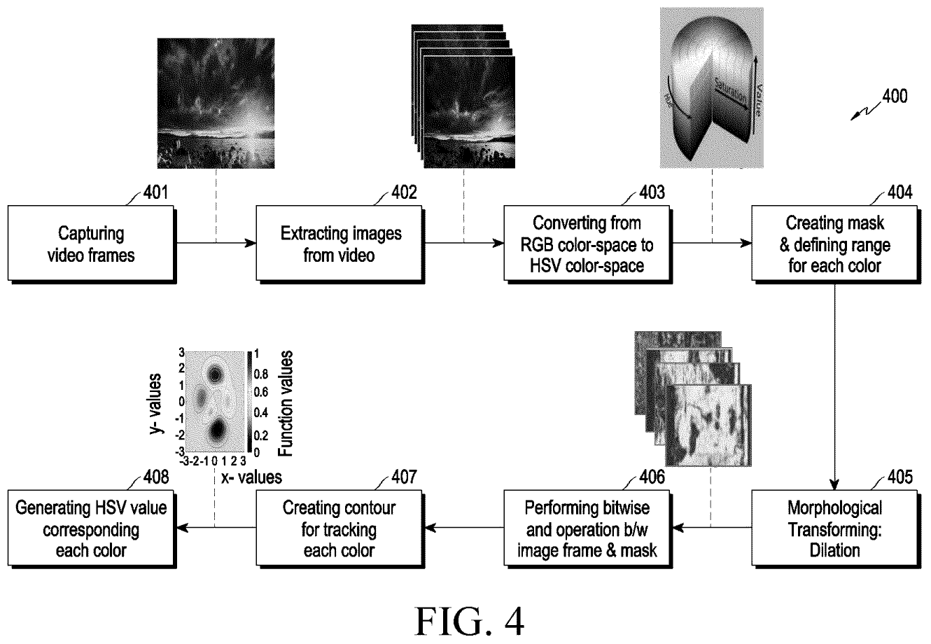

Referring to FIG. 3, at operations 300-301, the content feature extractor 242 may extract the one or more content features from the input frames (i.e., the plurality of first image frames) by utilizing one or more sub-modules, as described in conjunction with FIGS. 4 to 6. The one or more sub-modules may include a content hue detection unit 242a, a content contrast detection unit 242b, a content depth detection unit 242c, and a spatial focus demand estimating unit 242d. Further, the content feature extractor 242 may transfer the one or more extracted content features to the refractive error generator 245 for further processing.

The content hue detection unit 242a is configured to analyze a hue and color distribution (dominant color) within the input frames, identifying key color characteristics and patterns that contribute to the overall content of the images. The content contrast detection unit 242b is configured to evaluate one or more contrast levels within the input frames, and measure one or more differences in brightness, sharpness, and edges to extract information about the visual composition and highlighting of important elements. The content depth detection unit 242c is configured to assess a depth and three-dimensional structure of the content within the input frames, utilizing techniques such as stereoscopic analysis or depth mapping to extract spatial information about the scene. The spatial focus demand estimating unit 242d is configured to estimate a level of spatial focus (spatial focus time) and attention required to process the content within the input frames, providing insights into the visual complexity and the cognitive resources needed to comprehend the information presented in the input frames.

At operations 300-302, the user gesture recognizer 243 may extract the one or more user gesture features from the input frames (i.e., the plurality of first image frames), as described in conjunction with FIG. 7. Further, the user gesture recognizer 243 may transfer the one or more extracted user gesture features to the refractive error generator 245 for further processing.

For instance, in a digital presentation or interactive whiteboard setting, the user gesture recognizer 243 may extract hand gestures and body movements from the input frames to enable intuitive control and navigation of the presentation software. The user gesture recognizer 243 may detect a swiping gesture to advance slides, a pinching gesture to zoom in/out, or a pointing gesture to highlight specific content on the screen.

For instance, in an AR gaming or training application, the user gesture recognizer 243 may track the user's hand and body movements within the input frames to enable natural interaction with virtual objects or environments. The user gesture recognizer 243 may detect a grabbing gesture to pick up a virtual object, a throwing gesture to launch a projectile, or a pointing gesture to select a target in an AR scene.

For instance, in a smart home environment, the user gesture recognizer 243 may extract hand and body gestures from the input frames to allow users to control various smart devices and home appliances without physical contact. The user gesture recognizer 243 may detect a waving gesture to turn on/off lights, a circular motion to adjust the volume of a smart speaker, or a pointing gesture to change the channel on a smart TV.

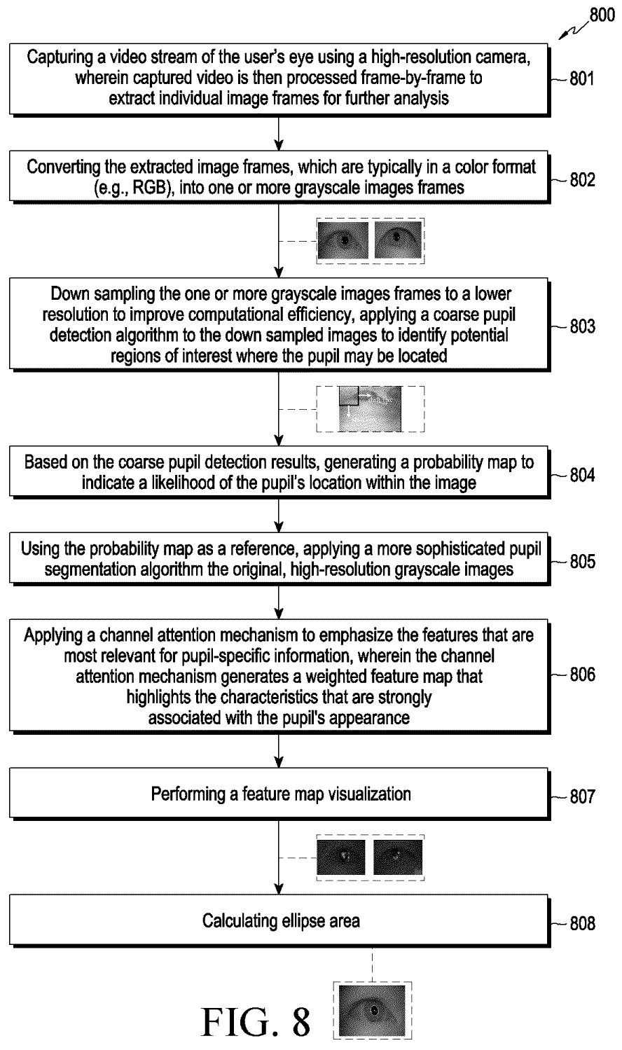

At operations 300, 303-304, the method includes triggering an eye analysis to generate the plurality of second image frames (i.e., ocular image) by utilizing a VR display rendering unit and an ocular image capturing unit. The ocular feature extractor 244 may then extract the one or more ocular features from the input frames by utilizing one or more sub-modules, as described in conjunction with FIGS. 8 to 12. The one or more sub-modules may include a pupil size determining unit 244a, a spatial focus time tracking unit 244b, an eye blinking rate calculating unit 244c, an eye-opening ratio determining unit 244d, and an eye's tear condition detection unit 244e. Further, the ocular feature extractor 244 may transfer the one or more extracted ocular features to the refractive error generator 245 for further processing.

At operation 305, the refractive error generator 245 may determine the refractive error experienced by the user while watching the plurality of generated first image frames through the VST-HMD device 200 based on the one or more content features, the one or more display features, the one or more user gesture features, and the one or more ocular features, as described in conjunction with FIG. 13. Further, the refractive error generator 245 may transfer the determined refractive error to the refractive risk score generator 246 for further processing.

At operation 306, the refractive risk score generator 246 may determine the refractive risk score (risk score) of the user based on the plurality of extracted first feature sets and the plurality of extracted second feature sets, and the determined refractive error, as described in conjunction with FIG. 14. Further, the refractive risk score generator 246 may transfer the determined refractive risk score to the virtual exercise adviser 249 and the target feature detector 247 for further processing.

At operation 307, the target feature detector 247 may determine the target feature variation vector based on the refractive risk score and the mean predicted refractive error, as described in conjunction with FIG. 15. The target feature detector 247 may include a target content features determining unit and a target display features determining unit. The target feature detector 247 may further extract one or more target content features and one or more target display features, and share them with the feature modifier 248 for further processing.

At operation 308, the feature modifier 248 may adjust, based on the one or more extracted target content features and the one or more extracted target display features, the one or more extracted content features and the one or more extracted display features for dynamically correcting the vision defect of the user by sending feedback to the input frames. The feature modifier 248 may adjust the one or more extracted content features and the one or more extracted display features, in order to mitigate the determined refractive risk score, for dynamically correcting the vision defect of the user.

At operation 309, the virtual exercise adviser 249 may recommend one or more personalized virtual training exercises for prolonged improvement of the vision defect of the user by utilizing the determined refractive risk score, as described in conjunction with FIG. 16.

FIG. 4 illustrates a scenario 400 where the content hue detection unit and the content contrast detection unit of the VST-HMD device execute one or more operations to extract the one or more content features (e.g., high-level semantic information, dominant color information, contrast information, etc.), according to an embodiment of the disclosure.

Referring to FIG. 4, in a scenario 400 where the content hue detection unit 242a and the content contrast detection unit 242b of the VST-HMD device 200 execute one or more operations to extract the one or more content features, at operations 401-402, the VST-HMD device 200 may extract one or more image frames (i.e., a plurality of first image frames) from a video stream, when the video stream is detected playing on the VST-HMD device 200. At operation 403, the VST-HMD device 200 then converts the one or more extracted image frames from a standard red, green, blue (RGB) color space to a hue, saturation, value (HSV) color space. This transformation allows for more efficient color analysis and manipulation. At operations 404-405, the VST-HMD device 200 creates masks and defines color ranges for each distinct color present in the one or more extracted image frames. These masked image frames are then subjected to morphological transformations, specifically dilation, to reduce noise and enhance the clarity of the color regions.

At operation 406, the VST-HMD device 200 then performs a bitwise AND operation between the one or more extracted image frames and the masks, effectively calculating the conjunction between the one or more extracted image frames and the masks. This operation retains the pixels with the same values while removing the pixels that do not match the defined color ranges. At operation 407, the VST-HMD device 200 then creates contours for each tracked color, generating a region-wise contour map that represents the distribution of the colors present in the frames. Finally, at operation 408, the VST-HMD device 200 generates the corresponding HSV values for each color identified in the one or more extracted image frames. This information allows the VST-HMD device 200 to accurately determine the features of the content (e.g., hue, saturation, brightness) being projected on the screen of the VST-HMD device 200 (display module).

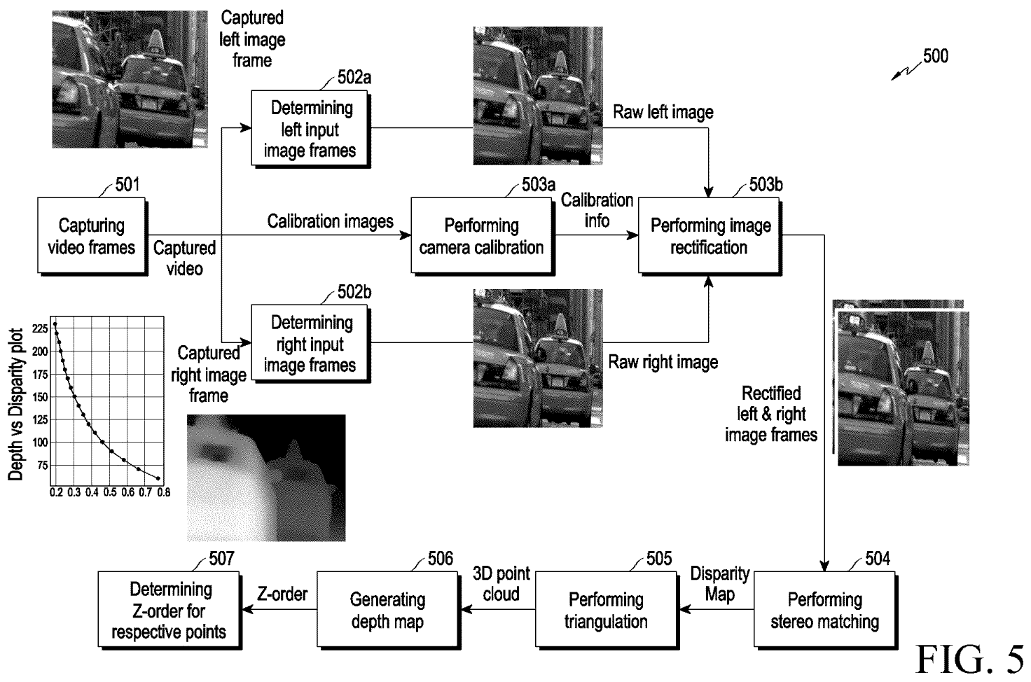

FIG. 5 illustrates a scenario where the content depth detection unit of the VST-HMD device executes one or more operations to extract the one or more content features (e.g., depth information), according to an embodiment of the disclosure.

Referring to FIG. 5, in a scenario 500 where the content depth detection unit 242c of the VST-HMD device 200 executes one or more operations to extract the one or more content features, the VST-HMD device 200 is equipped with a stereo depth perception system that enables the determination of the depth (z-order) of the content projected on the screen of the VST-HMD device 200.

At operations 501-502a/502b, from the captured video stream, the content depth detection unit 242c separates the one or more image frames corresponding to the left and right eyes (e.g., left input image frames and right input image frames). At operations 503a-503b and 504, these image frames are then calibrated and rectified to ensure that they are projected on the same plane, eliminating any disparity caused by the physical separation of the left and right image sensors. The rectified left and right image frames are then sent to a stereo-matching algorithm, which aligns the pixels between the two images and generates a disparity map. This disparity map represents the difference in the positions of the corresponding pixels between the left and right images, and it is directly proportional to the depth information of the scene.

At operations 505, 506, and 507, using the generated disparity map, the content depth detection unit 242c then computes a 3D point cloud, which represents the spatial coordinates of the various elements in the projected content. From the 3D point cloud, the content depth detection unit 242c may derive a depth map, which provides a detailed representation of the relative depths (z-order) of the different objects and regions within the projected content, as per Equation 1. The depth information obtained from this process enables the VST-HMD device 200 to accurately determine the spatial relationships and depth cues of the content being projected/displayed, allowing for more immersive and realistic virtual or augmented reality experiences.

$$z = \frac{f \times T}{d \times D} \qquad Equation 1$$

Where, Z=depth, d=size of a pixel in camera sensor, D=disparity, f=focal length of camera, and T=difference b/w left and right lens (image sensor)

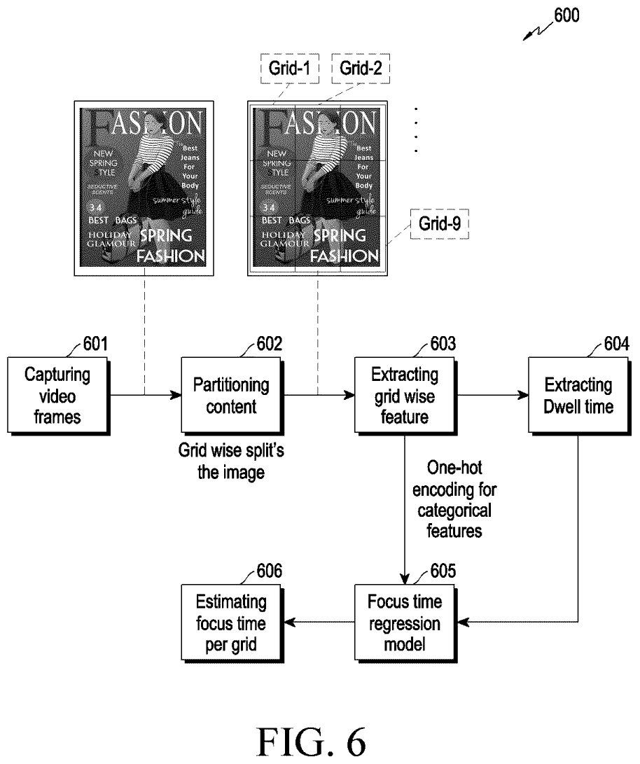

FIG. 6 illustrates a scenario where the spatial focus demand estimating unit of the VST-HMD device executes one or more operations to extract the one or more content features (e.g., spatial focus time information), according to an embodiment of the disclosure.

Referring to FIG. 6, in a scenario 600 where the spatial focus demand estimating unit 242d of the VST-HMD device 200 executes one or more operations to extract the one or more content features, at operations 601, the spatial focus demand estimating unit 242d may extract one or more image frames (i.e., the plurality of first image frames) from the video stream, when the video stream is detected playing on the VST-HMD device 200. At operation 602, the one or more extracted image frames are then divided into a grid-like structure, for example, effectively splitting a single image frame into nine distinct grids (e.g., grid-1, grid-2, . . . , grid-9). At operations 603, the spatial focus demand estimating unit 242d then may extract the one or more content features from each of the individual grids. These one or more extracted content features are categorized using a one-hot encoding technique, which allows for the effective representation of categorical data. Examples of the extracted features include text-based features and image-based features, as shown in Table 1.

TABLE 1

| | Text-based | | | | |
|---|---|---|---|---|---|
| | | | Presence | | |
| | Word | | of named | Image-based | |
| Grid | Count | Complexity | entity | Visibility | Complexity |
| Grid-1 | 1 | Medium | — | Large | Low |
| Grid-2 | 1 | Low | — | Large | Medium |
| Grid-3 | 15 | Medium | Magazine | Small | Low |

At operation 604, in addition to the content features, the spatial focus demand estimating unit 242d may also extract a dwell time associated with each grid, as shown in Table 2. This dwell time represents the duration for which the user's gaze or attention was focused on a particular section of the displayed content.

TABLE 2

| Grid | Dwell Time |
|---|---|
| 1 | Low |
| 2 | Medium |
| 3 | Low |

At operations 605-606, the spatial focus demand estimating unit 242d then may utilize, for example, a focus time regression model to estimate the focus time required for an ideal user to focus on the various sections of the displayed screen. This estimation is based on the combination of the one-hot encoded content features and the dwell time data, effectively determining the spatial focus demand (spatial focus time information), as shown in Table 3, which is the time required by the user to focus on different areas of the content based on factors such as complexity, visibility, and other content parameters.

TABLE 3

| | | | | | | | | | |
|---|---|---|---|---|---|---|---|---|---|
| Grid | 1 | 2 | 3 | 4 | 5 | 6 | 7 | 8 | 9 |
| Time | 3 | 2 | 3 | 6 | 1 | 5 | 5 | 5 | 4 |
| (sec) | | | | | | | | | |

By employing this advanced content analysis and focus time estimation (spatial focus time information), the VST-HMD device 200 may gain a deeper understanding of the user's visual attention and cognitive load, enabling more effective content optimization, user experience enhancement, and adaptive rendering strategies for the projected virtual or augmented reality scenarios.

FIG. 7 illustrates a scenario where the user gesture recognizer of the VST-HMD device executes one or more operations to extract the one or more user gesture features, according to an embodiment of the disclosure.

Referring to FIG. 7, in a scenario 700 where the user gesture recognizer 243 of the VST-HMD device 200 executes one or more operations to extract the one or more user gesture features, at operation 701, the user gesture recognizer 243 may extract one or more image frames (i.e., the plurality of first image frames) from the video stream, when the video stream is detected playing on the VST-HMD device 200. At operation 702, the user gesture recognizer 243 may perform a series of pre-processing operations on the one or more extracted image frames. These pre-processing operations may include resizing the image frames, converting the RGB color space to grayscale, and determining a region of interest (ROI) within the one or more extracted image frames, such as the user's hand. At operation 703, the user gesture recognizer 243 may then proceed to perform hand segmentation using a combination of skin color detection and background subtraction techniques. This allows the user gesture recognizer 243 to isolate the user's hand region from the rest of the scene, enabling more accurate gesture feature extraction.

At operation 704, the user gesture recognizer 243 may then extract various user gesture features from the isolated/segmented hand region. These user gesture features may include a number of key points, the distances, and angles between one or more key points, one or more location and size of the palm, and other relevant parameters. These user gesture features are then compiled into a comprehensive feature vector. At operation 705, using this comprehensive feature vector, the user gesture recognizer 243 may perform a classification task to determine the probability distribution over various gesture classes, such as “zoom-in,” “zoom-out,” and so on, as shown in Table 4.

TABLE 4

| Gesture | Zoom in | Zoom out | Scroll up | Many more . . . |
|---|---|---|---|---|
| Probability | 0.85 | 0.1 | 0.03 | 0.02 |

At operation 706, the user gesture recognizer 243 may then make a decision based on the determined probability distribution. For instance, if the highest probability of a particular gesture class exceeds a predefined threshold, the user gesture recognizer 243 triggers the corresponding functionality. If the probability distribution does not meet the threshold, the user gesture recognizer 243 continues to the next video frame/video stream, maintaining a continuous evaluation of the user's gestural inputs. This integrated approach of image preprocessing, hand segmentation, feature extraction, and probabilistic classification enables the VST-HMD device 200 to accurately interpret the user's gestural commands (one or more user gesture features), allowing for more intuitive and natural interactions within the virtual or augmented reality environment.

FIG. 8 is a flow diagram illustrating a method for determining the one or more ocular features (e.g., pupil size information) by the pupil size determining unit of the VST-HMD device, according to an embodiment of the disclosure.

Referring to FIG. 8, in a method 800 for determining the one or more ocular features (e.g., pupil size information) by the pupil size determining unit 244a of the VST-HMD device 200, the method 800 may execute multiple operations to determine the one or more ocular features, which are given below.

At operation 801, the method 800 includes capturing the video stream of the user's eye using a high-resolution camera (camera module), wherein captured video (i.e., plurality of second image frames) is then processed frame-by-frame to extract individual image frames for further analysis. At operation 802, the method 800 includes converting the extracted image frames, which are typically in a color format (e.g., RGB), into one or more grayscale image frames. This conversion reduces the dimensionality of the image data, simplifying subsequent processing steps and reducing computational complexity.

At operation 803, the method 800 includes down-sampling the one or more grayscale image frames to a lower resolution to improve computational efficiency, applying a coarse pupil detection algorithm to the down-sampled images to identify potential regions of interest where the pupil may be located. This operation 803 provides a rough estimate of the pupil's location, which is then used as a starting point for more refined pupil segmentation.

At operation 804, the method 800 includes generating a probability map to indicate a likelihood of the pupil's location within the image based on the coarse pupil detection results. The probability map serves as a guide for the subsequent refinement operation, highlighting the areas where the pupil is most likely to be found. At operation 805, the method 800 includes applying a more sophisticated pupil segmentation algorithm to the original, high-resolution grayscale images using the probability map as a reference. This refinement operation aims to accurately delineate the boundaries of the pupil, providing a high-precision segmentation mask. At operation 806, the method 800 includes applying a channel attention mechanism to emphasize the features that are most relevant for pupil-specific information, wherein the channel attention mechanism generates a weighted feature map that highlights the characteristics that are strongly associated with the pupil's appearance.

At operation 807, the method 800 includes performing a feature map visualization. In other words, the feature map visualization is visually represented to provide insights into the specific features that the pupil size determining unit 244a has identified as being most important for pupil detection and segmentation. This feature map visualization can be useful for understanding a decision-making process of the pupil size determining unit 244a and for further improving the algorithms. At operation 808, the method 800 includes calculating the ellipse area. In other words, a segmented pupil region is approximated by an ellipse, whose major and minor axes (a and b, respectively) are measured. The area of the ellipse is then calculated using below mentioned Equation 2.

$$A = a * b * π \qquad Equation 2$$

This calculated ellipse area serves as a ground truth for the pupil size, which can be compared to other methods or used for further analysis, for example, using below-mentioned Equation 3.

$$mean error = \frac{1}{n} \sum \frac{\vert prediction ( pix ) - ground truth ( pix ) \vert}{ground truth ( pix )} \times 100 \% \qquad Equation 3$$

4

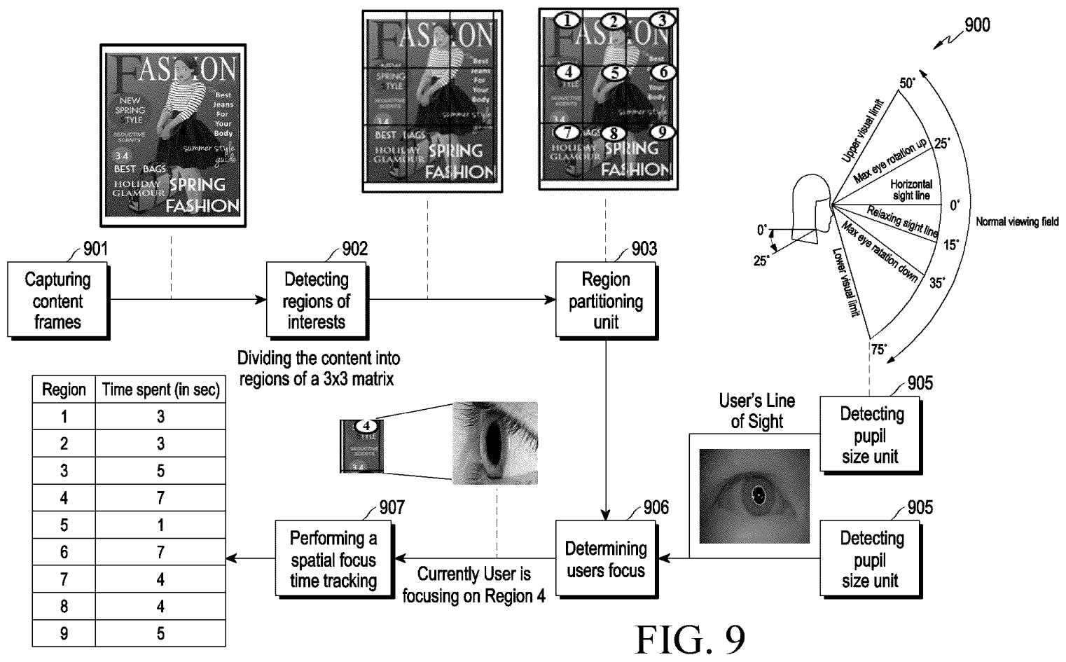

FIG. 9 illustrates a scenario where the spatial focus time tracking unit of the VST-HMD device executes one or more operations to extract the one or more ocular features (e.g., spatial focus time matrix information), according to an embodiment of the disclosure.

Referring to FIG. 9, in a scenario 900, where the spatial focus time tracking unit 244b of the VST-HMD device 200 executes one or more operations to extract the one or more ocular features, at operations 901, the spatial focus time tracking unit 244b may extract one or more image frames (i.e., the plurality of second image frames), when a video stream is detected playing on the VST-HMD device 200. At operations 902-903, these one or more extracted image frames are then divided into a grid-like structure, for example, effectively splitting a single image frame into nine distinct grids (e.g., grid-1 as “(\textcircled{1}), grid-2 as “\textcircled{2}”, . . . , grid-9 as “\textcircled{9}”) by utilizing an ROI detection mechanism and a region partitioning unit.

At operations 904, the spatial focus time tracking unit 244b may determine information associated with a user's line of sight by detecting data related to their normal viewing field. The normal viewing field can be characterized by, for example, an upper visual limit (50 degrees), maximum eye rotation up (25 degrees), horizontal sight line (0 degrees), relaxed sight line (15 degrees), maximum eye rotation down (35 degrees), and a lower visual limit (75 degrees).

At operations 905, 906, and 907, the spatial focus time tracking unit 244b may utilize the pupil size determining unit 244a to gather the pupil size information, which can be used to infer the user's focus and perform spatial focus time tracking. The time spent by the user focusing on different regions (grids) of the visual field is calculated based on the type and amount of data present in each region, combined with the user's pupil size and line of sight. By employing this advanced content analysis and focus time estimation (spatial focus time information), the VST-HMD device 200 can gain a deeper understanding of the user's visual attention and cognitive load. This enables more effective content optimization, user experience enhancement, and adaptive rendering strategies for the projected virtual or augmented reality scenarios.

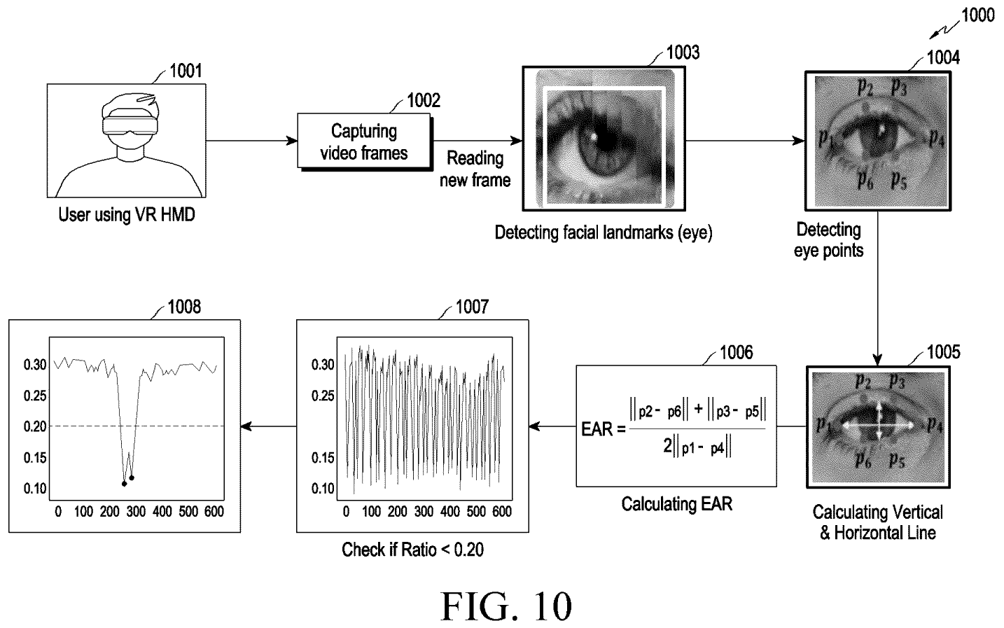

FIG. 10 illustrates a scenario where the eye blinking rate calculating unit of the VST-HMD device executes one or more operations to extract the one or more ocular features (e.g., blinking rate information), according to an embodiment of the disclosure.

Referring to FIG. 10, in a scenario 1000, where the eye blinking rate calculating unit 244c of the VST-HMD device 200 executes one or more operations to extract the one or more ocular features, at operations 1001-1002, the eye blinking rate calculating unit 244c may extract one or more image frames (i.e., the plurality of second image frames), when a video stream is detected playing on the VST-HMD device 200. At operations 1003-1004, the eye blinking rate calculating unit 244c may utilize computer vision techniques to detect and extract facial landmarks, such as the eyes, from the one or more extracted image frames. Upon successfully detecting the facial landmarks, the eye blinking rate calculating unit 244c may detect six specific eye points (p1 through p6) corresponding to an eye region. At operations 1005-1006, based on the detected eye points, the eye blinking rate calculating unit 244c may construct two reference lines—a vertical line and a horizontal line. These lines serve as the basis for calculating an Eye Aspect Ratio (EAR), a well-established metric used to infer eye blink events (e.g., EAR will be almost equal to 0 when a blink occurs). The EAR is computed as the ratio between the vertical and horizontal distances of the eye landmarks.

At operations 1007-1008, when the EAR falls below a predefined threshold (e.g., 0.2), the eye blinking rate calculating unit 244c may interpret this as a blink event. The eye blinking rate calculating unit 244c may then track the blink occurrences by monitoring the transitions of the EAR, where a previous EAR above 0.2 followed by a current EAR below 0.2 is indicative of a blink. This eye blink detection mechanism allows the eye blinking rate calculating unit 244c to closely monitor the user's eye movements and blinking patterns, which can provide valuable insights into the user's visual engagement, cognitive state, and potential fatigue or drowsiness levels. The extracted eye-related data can be further leveraged to optimize the user experience, adapt content rendering, and enable more intelligent and responsive interactions within the virtual or augmented reality environment.

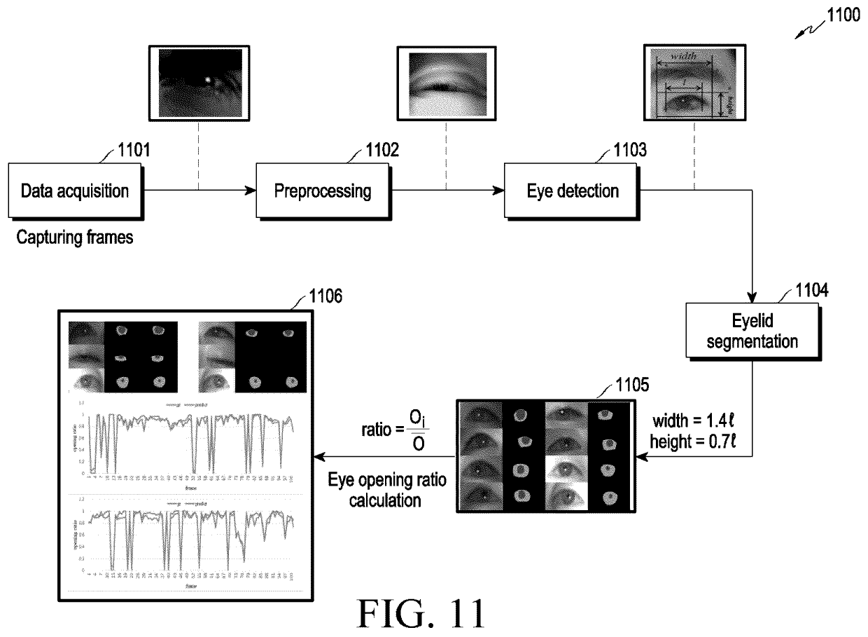

FIG. 11 illustrates a scenario where the eye-opening ratio determining unit of the VST-HMD device executes one or more operations to extract the one or more ocular features (e.g., eye-opening size pattern information), according to an embodiment of the disclosure.

Referring to FIG. 11, in a scenario 1100, where the eye-opening ratio determining unit 244d of the VST-HMD device 200 executes one or more operations to extract the one or more ocular features, at operations 1101-1102, the eye-opening ratio determining unit 244d may extract one or more image frames (i.e., the plurality of second image frames), when a video stream is detected playing on the VST-HMD device 200. The eye-opening ratio determining unit 244d may include a preprocessing module, the preprocessing module is configured to receive the one or more extracted image frames from the user's viewpoint. The preprocessing module is configured to perform standard image pre-processing operations, such as resizing, color space conversion, and noise reduction, to prepare the images for further analysis. The preprocessed image frames are then passed on to the next module for eye detection.

At operation 1103, the eye-opening ratio determining unit 244d may include an eye detection module, the eye detection module is configured to utilize computer vision algorithms, such as deep learning-based models, to detect the presence and location of the user's eyes within the one or more extracted image frames (input image frames). It identifies the ROIs containing the eyes and extracts the relevant eye-related data for further processing. The detected eye regions are then forwarded to an eyelid segmentation module of the eye-opening ratio determining unit 244d.

At operation 1104, the eyelid segmentation module is configured to employ image segmentation techniques, such as edge detection, thresholding, or semantic segmentation models, to precisely delineate the boundaries of the upper and lower eyelids. This module focuses on segmenting the eyelids from the detected eye regions. The segmented eyelid regions are passed on to an opening and closing detection module of the eye-opening ratio determining unit 244d.

At operation 1105, the opening and closing detection module is configured to analyze one or more changes in the eyelid positions over time to detect eye-opening and closing events. The opening and closing detection module is further configured to compare the segmented eyelid regions across consecutive frames and identifies the frames where the eye is transitioning from open to closed or vice versa. The detected opening and closing events are then used to calculate an eye-opening ratio in the next module.

At operation 1106, the eye-opening ratio determining unit 244d may include an eye opening ratio (EOR) calculation module, the eye opening ratio calculation module is configured to take the detected eye opening and closing events from the previous module and computes the EOR. The EOR is a metric that represents the proportion of time the eye is open during a given time period. The EOR calculation module calculates the EOR by analyzing the duration of the eye's open and closed states and expressing it as a ratio. The computed EOR is then made available for further analysis and applications, such as user engagement monitoring, fatigue detection, or adaptive rendering in virtual/augmented reality environments.

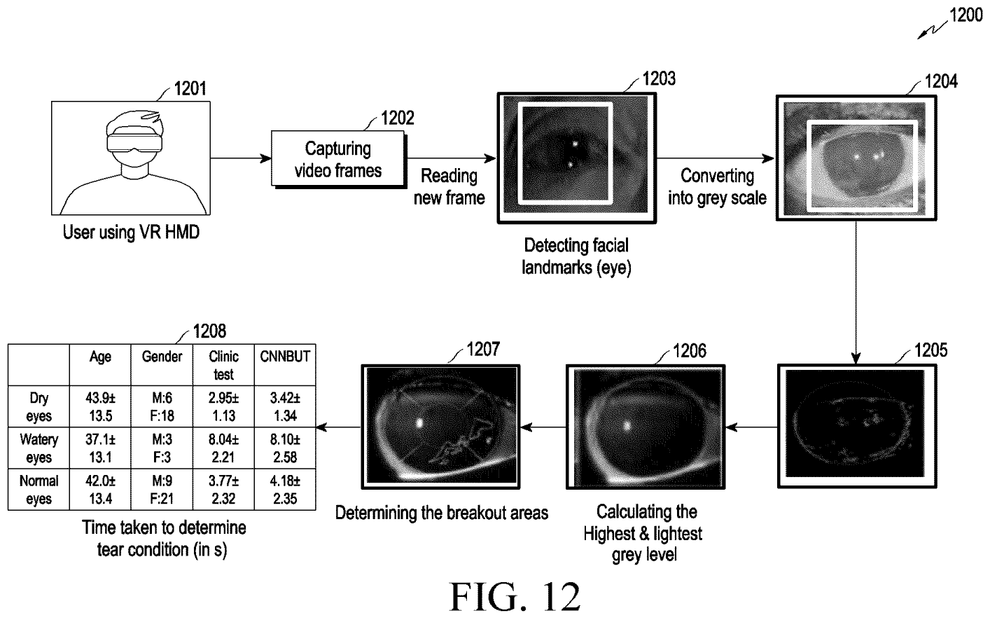

FIG. 12 illustrates a scenario where the eye's tear condition detection unit of the VST-HMD device executes one or more operations to extract the one or more ocular features (e.g., eye tear condition information), according to an embodiment of the disclosure.

Referring to FIG. 12, in a scenario 1200, where the eye's tear condition detection unit 244e of the VST-HMD device 200 executes one or more operations to extract the one or more ocular features, at operations 1201-1202, the eye's tear condition detection unit 244e may extract one or more image frames (i.e., the plurality of second image frames), when a video stream is detected playing on the VST-HMD device 200. At operations 1203-1204, the eye's tear condition detection unit 244e may utilize computer vision techniques to detect and extract facial landmarks, such as the eyes, from the one or more extracted image frames. Upon successfully detecting the facial landmarks, the eye's tear condition detection unit 244e may convert the one or more extracted image frames (captured image frames) into grayscale format to simplify the processing and feature extraction. The eye's tear condition detection unit 244e may then perform an eye size identification to detect the ROI containing the eye within the one or more extracted image frames.

At operation 1205, the eye's tear condition detection unit 244e may crop an edge map of an iris region, removing any outliers, such as eyelashes or eyelids, that may interfere with the analysis. This operation helps to isolate the iris region for further processing. At operation 1206, the eye's tear condition detection unit 244e may calculate the highest and lightest gray level values within the iris region. At the start of the capturing process, the eye's tear condition detection unit 244e may record the highest and lowest gray level values and deem them as the initial threshold values.

At operation 1207, the eye's tear condition detection unit 244e may then determine one or more “breakout areas” within the iris region. Areas having a higher gray level value than the established threshold are considered “dry,” while those with a lighter gray level are deemed “moist/watery.” At operation 1208, the eye's tear condition detection unit 244e may calculate a time taken to determine the tear condition (in seconds). This time metric represents a duration required for the eye's tear condition detection unit 244e to assess the eye tear condition information, classifying it as dry eyes, watery eyes, or normal eyes, as shown in a tabular format. The extracted information (eye tear condition information) can be further utilized for applications such as dry eye detection, eye fatigue monitoring, and personalized user experience optimization in various scenarios, including virtual and augmented reality environments.

FIG. 13 illustrates a scenario where the refractive error generator of the VST-HMD device executes one or more operations to determine the refractive error experienced by the user while watching the plurality of generated first image frames through the VST-HMD device, according to an embodiment of the disclosure.

Referring to FIG. 13, in a scenario 1300, where the refractive error generator 245 of the VST-HMD device 200 executes one or more operations to determine the refractive error experienced by the user while watching the plurality of generated first image frames through the VST-HMD device 200, at operation 1301, the refractive error generator 245 may determine a temporal feature vector (temporal encoded input) for each of the plurality of generated first image frames and the plurality of generated second image frames by utilizing a pre-trained autoregressive neural network based on the plurality of extracted first feature sets and the plurality of extracted second feature sets. The plurality of extracted first feature sets (e.g., hue, brightness, gesture, etc.) are received from the content feature extractor 242 and the user gesture recognizer 243. The plurality of extracted second feature sets (e.g., pupil size, eye-opening size, blinking rate, etc.) are received from the ocular feature extractor 244. The pre-trained autoregressive neural network captures one or more temporal dynamics and dependencies present in a sequence of image frames, allowing for the extraction of a robust temporal representation.

At operation 1302, the refractive error generator 245 may determine a Three-dimensional (3D) feature vector for each of the plurality of generated second image frames (video frames) by utilizing a pre-trained multi-layer perceptron (MLP) neural network based on the one or more extracted ocular features. The 3D feature vector provides a compact and informative encoding of ocular-specific information present in each of the plurality of generated second image frames. The pre-trained MLP neural network captures one or more intricate spatial and structural characteristics of an ocular region (e.g., iris region, eye region, eyelid region, etc.), enabling a robust representation of the one or more extracted ocular features.

At operation 1303, the refractive error generator 245 may include a feature concatenation unit, and the feature concatenation unit is configured to combine the determined temporal feature vector and the determined 3D feature vector into a single feature input vector. At operations 1304-1305, the feature concatenation unit is configured to feed the single feature input vector into a pre-trained MLP classifier (feed-forward classifier) to determine a type of refractive error based on a predefined threshold value. The type of refractive error may include, but is not limited to, myopia, hyperopia, astigmatism, and normal refractive status, as shown in a tabular format.

FIG. 14 illustrates a scenario where the refractive risk score generator of the VST-HMD device executes one or more operations to determine the refractive risk score of the user, according to an embodiment of the disclosure.

Referring to FIG. 14, in a scenario 1400, where the refractive risk score generator 246 of the VST-HMD device 200 executes one or more operations to determine the refractive risk score of the user, at operations 1401-1402, the refractive risk score generator 246 may obtain a determined refractive error for a predefined set of consecutive image frames (e.g., frame set 1, frame set 2, . . . , frame set k) from each of the plurality of generated first image frames and the plurality of generated second image frames. The refractive risk score generator 246 may determine, upon obtaining the determined refractive error for the predefined consecutive frame set, a mean distribution of refractive errors by averaging distributions of the determined refractive error values obtained for the predefined set of consecutive image frames. The refractive risk score generator 246 may determine a mean refractive error by selecting a refractive error class with maximum probability in the determined mean distribution of refractive errors.

At operation 1403, the refractive risk score generator 246 may feed the determined mean distribution of refractive errors and the mean refractive error as input to a pre-trained long short-term memory (LSTM) neural network module to determine the refractive risk score of the user. The refractive risk score generator 246 may store the determined refractive risk score in the VST-HMD device 200. The refractive risk score generator 246 may generate a risk profile of the user based on the stored refractive risk score. At operations 1404-1405, the refractive risk score generator 246 may include a former risk score comparator, the former risk score comparator determines whether a current risk score is lower than a previous risk score (stored refractive risk score). At operation 1406, the refractive risk score generator 246 may not perform any operation to adjust the current display feature. At operation 1407, the refractive risk score generator 246 may perform one or more operations to modify the target display feature, as described in conjunction with FIG. 15.

FIG. 15 illustrates a scenario where the target feature detector of the VST-HMD device executes one or more operations to determine the target features variation vector, according to an embodiment of the disclosure.

Referring to FIG. 15, in a scenario 1500, where the target feature detector 247 of the VST-HMD device 200 executes one or more operations to determine the target features variation vector, at operation 1501, the target feature detector 247 may include a data calculating unit, the data calculating unit is configured to generate a target feature delta vector associated with the one or more content features and the one or more display features based on the determined refractive risk score (refractive risk score frameset). At operation 1502, the target feature detector 247 may include a co-efficient determining unit, the co-efficient determining unit is configured to generate a target feature co-efficient vector associated with the one or more content features and the one or more display features based on the determined mean of refractive error. At operation 1503, the target feature detector 247 may include a target features combination unit, the target features combination unit is configured to combine the generated target feature delta vector and the generated target feature co-efficient vector to generate the target feature variation vector, which may utilize to dynamically adjust at least one of the one or more content features and the one or more display features based on the generated target feature variation vector.

FIG. 16 illustrates one or more scenarios associated with the virtual exercise adviser of the VST-HMD device, according to an embodiment of the disclosure.

Referring to FIG. 16, the virtual exercise adviser 249 of the VST-HMD device 200 may recommend the one or more personalized virtual training exercises for prolonged improvement of the vision defect of the user. Examples of the one or more personalized virtual training exercises may include, but are not limited to, a virtual tic-tac-toe exercise (1601, 1602, and 1603), a virtual focus exercise (1604 and 1605), a virtual swinging exercise (1606 and 1607), and a virtual rotating exercise (1608).

In the virtual tic-tac-toe exercise (1601, 1602, and 1603), the virtual exercise adviser 249 gradually moves an object, such as a car, farther and farther away from the user. The user is prompted to indicate when the object becomes blurred or appears doubled. Once the user provides feedback (e.g., voice command as “stop”), the virtual exercise adviser 249 then displays a virtual tic-tac-toe board at the corresponding depth and asks the user to finger-point at specific grids. This exercise may be performed at least once a day, and the user may notice a gradual improvement in their vision over time.

In the virtual focus exercise (1604 and 1605), the virtual exercise adviser 249 asks the user to maintain focus on a specific object, such as a car. The virtual exercise adviser 249 then displays a car with a lower wavelength color (e.g., yellow) at a closer depth (d1) and suddenly changes it to a higher wavelength color (e.g., red) at a greater depth (d2). This sequence is repeated a few times. Performing this process 10 times per day can help improve the user's ability to focus on objects at farther distances.

In the virtual swinging exercise (1606 and 1607), the virtual exercise adviser 249 asks the user to focus on an object, such as a car, simulated at a long depth (d). The virtual exercise adviser 249 then slowly moves the object sideways, both left and right. During this exercise, the user is instructed to blink regularly to keep the eyes moist and clean, as this can help alleviate any eye strain.

In the virtual rotating exercise (1608), the virtual exercise adviser 249 asks the user to focus on an object, such as a car, simulated at a long depth (d). The virtual exercise adviser 249 then slowly rotates the object in both clockwise and counterclockwise directions, for a total duration of approximately one minute. This exercise helps to keep the user's eyes lubricated and can also help alleviate eye strain.

The aforementioned exercises are designed to gradually improve the user's visual acuity, depth perception, and eye-muscle coordination, which can be beneficial for various applications, including virtual and augmented reality experiences.

FIG. 17 illustrates a scenario where the feature modifier of the VST-HMD device executes one or more operations to adjust one or more extracted content features and one or more extracted display features for dynamically correcting the vision defect of the user, according to an embodiment of the disclosure.

Referring to FIG. 17, consider a scenario where one or more image frames are displayed on the screen of the VST-HMD device 200. The VST-HMD device 200 may perform a plurality of operations to determine the refractive error and refractive risk score of the user. A detailed description related to the plurality of operation is covered in the description related to FIGS. 2 to 16, and is omitted herein for the sake of brevity. Upon determining the refractive error and refractive risk score of the user, the feature modifier 248 of the VST-HMD device 200 may adjust the one or more extracted content features and the one or more extracted display features for dynamically correcting the vision defect of the user based on at least one of the determined refractive error and the determined refractive risk score of the user, in order to mitigate the determined refractive risk score, for dynamically correcting the vision defect of the user.

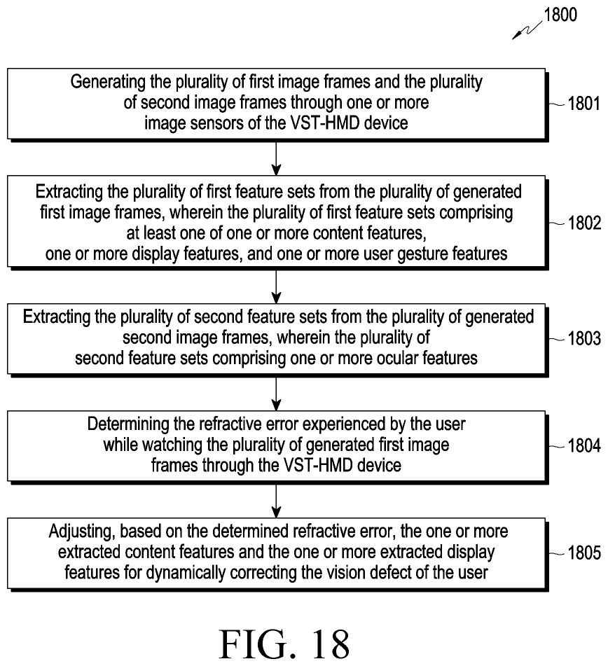

FIG. 18 is a flow diagram illustrating a method for dynamically correcting the vision defect of the user using the VST-HMD device, according to an embodiment of the disclosure.

Referring to FIG. 18, a method 1800 may execute multiple operations to dynamically correct the vision defect of the user, which are given below.

At operation 1801, the method 1800 includes generating the plurality of first image frames and the plurality of second image frames through one or more image sensors of the VST-HMD device 200. At operation 1802, the method 1800 includes extracting the plurality of first feature sets from the plurality of generated first image frames, wherein the plurality of first feature sets comprising at least one of the one or more content features, the one or more display features, and the one or more user gesture features. At operation 1803, the method 1800 includes extracting the plurality of second feature sets from the plurality of generated second image frames, wherein the plurality of second feature sets comprising one or more ocular features. At operation 1804, the method 1800 includes determining the refractive error experienced by the user while watching the plurality of generated first image frames through the VST-HMD device 200. At operation 1805, the method 1800 includes adjusting, based on the determined refractive error, the one or more extracted content features and the one or more extracted display features for dynamically correcting the vision defect of the user. Further, a detailed description related to the various operations of FIG. 18 is covered in the description related to FIGS. 2 to 16, and is omitted herein for the sake of brevity.

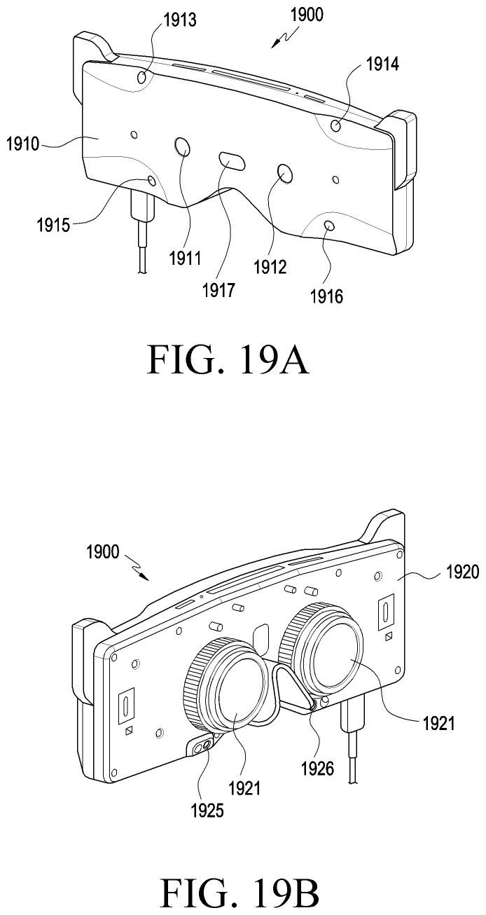

FIGS. 19A and 19B are diagrams illustrating a wearable device 900 (e.g., the VST-HMD device 200) according to various embodiments of the disclosure.

Referring to FIGS. 19A and 19B, in an embodiment, camera modules 1911, 1912, 1913, 1914, 1915, and 1916 and/or a depth sensor 1917 for obtaining information related to the surrounding environment of the wearable device 1900 may be disposed on a first surface 1910 of the housing. In an embodiment, the camera modules 1911 and 1912 may obtain an image related to the surrounding environment of the wearable device. In an embodiment, the camera modules 1913, 1914, 1915, and 1916 may obtain an image while the wearable device is worn by the user. Images obtained through the camera modules 1913, 1914, 1915, and 1916 may be used for simultaneous localization and mapping (SLAM), 6 degrees of freedom (6DoF), 3 degrees of freedom (3DoF), subject recognition and/or tracking, and may be used as an input of the wearable electronic device by recognizing and/or tracking the user's hand. In an embodiment, the depth sensor 1917 may be configured to transmit a signal and receive a signal reflected from a subject, and may be used to identify the distance to an object, such as time of flight (TOF). According to an embodiment, face recognition camera modules 1925 and 1926 and/or a display 1921 (and/or a lens) may be disposed on the second surface 1920 of the housing. In an embodiment, the face recognition camera modules 1925 and 1926 adjacent to the display may be used for recognizing a user's face or may recognize and/or track both eyes of the user. In an embodiment, the display 1921 (and/or lens) may be disposed on the second surface 1920 of the wearable device 1900. In an embodiment, the wearable device may not include the camera modules 1915 and 1916 among a plurality of camera modules 1913, 1914, 1915, and 1916. As described above, the wearable device according to an embodiment may have a form factor for being worn on the user's head. The wearable device may further include a strap for being fixed on the user's body and/or a wearing member (e.g., the wearing member). The wearable device may provide a user experience based on augmented reality, virtual reality, and/or mixed reality within a state worn on the user's head. The disclosed method and/or the VST-HMD device 200 has several advantages over the existing VST-HMD device, which are stated below.

The disclosed method provides a software-based solution that adjusts the displayed/projected image frame to align with the user's refractive error (refractive index). This eliminates the need for external hardware, such as vision correction lenses or glasses, to be used with the VST-HMD device 200. This results in a more economical and streamlined solution for the user, providing a clear and immersive experience.

The disclosed method dynamically adjusts the focal length of the content and display features based on the determined refractive error and/or refractive risk score of the user. This enables the VST-HMD device 200 to effectively correct the user's vision defects, delivering a more personalized and optimized visual experience.

The disclosed method includes a personalized virtual training exercise feature that helps improve the user's eyesight condition over prolonged use. This feature detects eye strain and automatically triggers a virtual eye training exercise, allowing the user to take breaks and engage in targeted exercises to relieve eye strain. After the exercise, the VST-HMD device 200 seamlessly resumes the previously ongoing content, providing a comprehensive and user-centric solution.

The various actions, acts, blocks, steps, or the like in the flow diagrams may be performed in the order presented, in a different order, or simultaneously. Further, in some embodiments, some of the actions, acts, blocks, steps, or the like may be omitted, added, modified, skipped, or the like without departing from the scope of the disclosure.

Unless otherwise defined, all technical and scientific terms used herein have the same meaning as commonly understood by one ordinary skilled in the art to which this disclosure belongs. The system, methods, and examples provided herein are illustrative only and not intended to be limiting.

While specific language has been used to describe the present subject matter, any limitations arising on account thereto, are not intended. As would be apparent to a person in the art, various working modifications may be made to the method to implement the inventive concept as taught herein. The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment.

The embodiments disclosed herein can be implemented using at least one hardware device and performing network management functions to control the elements.

It will be appreciated that various embodiments of the disclosure according to the claims and description in the specification can be realized in the form of hardware, software or a combination of hardware and software.

Any such software may be stored in non-transitory computer readable storage media. The non-transitory computer readable storage media store one or more computer programs (software modules), the one or more computer programs include computer-executable instructions that, when executed by one or more processors of an electronic device individually or collectively, cause the electronic device to perform a method of the disclosure.

Any such software may be stored in the form of volatile or non-volatile storage such as, for example, a storage device like read only memory (ROM), whether erasable or rewritable or not, or in the form of memory such as, for example, random access memory (RAM), memory chips, device or integrated circuits or on an optically or magnetically readable medium such as, for example, a compact disk (CD), digital versatile disc (DVD), magnetic disk or magnetic tape or the like. It will be appreciated that the storage devices and storage media are various embodiments of non-transitory machine-readable storage that are suitable for storing a computer program or computer programs comprising instructions that, when executed, implement various embodiments of the disclosure. Accordingly, various embodiments provide a program comprising code for implementing apparatus or a method as claimed in any one of the claims of this specification and a non-transitory machine-readable storage storing such a program.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. A method for controlling a wearable device, comprising:
generating at least one first image frame for providing a virtual reality or an augmented reality through the wearable device;
obtaining, through at least one camera of the wearable device, at least one second image frame of an ocular of the user;
identifying at least one first feature from the at least one first image frame, wherein the at least one first feature comprises at least one of one or more content features or one or more display features, and one or more user gesture features;
identifying at least one second feature from the at least one second image frame, wherein the at least one second feature comprises one or more ocular features of the user;
based on the at least one first feature and the at least one second feature, determining a type of a refractive error of the user while the virtual reality or the augmented reality is provided;
adjusting, based on the type of the refractive error, at least one of the identified content features or the identified display features, and
based on the at least one of the adjusted content features or the adjusted display features, providing the virtual reality or the augmented reality.

2. The method of claim 1, further comprising:
determining a refractive risk score, wherein the refractive risk score is determined based on the identified first feature, the identified second feature and the type of the refractive error, and
adjusting the one or more content features and the one or more display features, in order to mitigate the determined refractive risk score, for dynamically correcting the refractive error of the user.

3. The method of claim 1, further comprising:
determining, based on the first feature and the second feature, a temporal feature vector for the generated at least one first image frame and the obtained at least one second image frame by utilizing a pre-trained autoregressive neural network;
determining, based on the one or more ocular features, a three-dimensional (3D) feature vector for the obtained at least one second image frame by utilizing a pre-trained multi-layer perceptron (MLP) neural network;
combining the determined temporal feature vector and the determined 3D feature vector into a single feature input vector, and
feeding the single feature input vector into a pre-trained MLP classifier to determine the type of the refractive error based on a predefined threshold value.

4. The method of claim 3,
wherein the pre-trained autoregressive neural network captures one or more temporal dynamics and dependencies present in a sequence of image frames, allowing for the extraction of a robust temporal representation,
wherein the pre-trained MLP neural network captures one or more intricate spatial and structural characteristics of an ocular region, enabling a robust representation of the one or more extracted ocular features,
wherein the 3D feature vector provides a compact and informative encoding of ocular-specific information present in the obtained at least one second image frame, and
wherein the type of the refractive error comprises at least one of myopia, hyperopia, astigmatism, and normal refractive status.

5. The method of claim 2, further comprising:
obtaining the type of refractive error for a predefined set of consecutive image frames from the generated at least one first image frame and the obtained at least one second image frame;
determining, upon obtaining the type of the refractive error for the predefined set of consecutive image frames, a mean distribution of refractive errors by averaging distributions of determined refractive error values obtained for the predefined set of consecutive image frames;
determining a mean refractive error by selecting a refractive error class with maximum probability in the determined mean distribution of refractive errors; and
feeding the determined mean distribution of refractive errors and the mean refractive error as input to a pre-trained long short-term memory (LSTM) neural network module to determine the refractive risk score.

6. The method of claim 5, further comprising:
storing the determined refractive risk score in the wearable device, and generating a risk profile of the user based on the stored refractive risk score.

7. The method of claim 1, further comprising:
generating, based on the determined refractive risk score, a target feature delta vector associated with the one or more content features and the one or more display features;
generating, based on the determined mean of refractive error, a target feature co-efficient vector associated with the one or more content features and the one or more display features;
combining the generated target feature delta vector and the generated target feature co-efficient vector to generate a target feature variation vector, and dynamically adjusting at least one of the one or more content features and the one or more display features based on the generated target feature variation vector.

8. The method of claim 1, further comprising:
recommending one or more personalized virtual training exercises for prolonged improvement of the refractive error of the user.

9. The method of claim 1, wherein the one or more content features represent a characteristic of visual content depicted in the generated at least one first image frame comprising at least one of object recognition information, scene classification information, text identification information, high-level semantic information, dominant color information, contrast information, depth information, or spatial focus time information.

10. The method of claim 1, wherein the one or more display features represent a characteristic of a visual presentation and layout of the visual content depicted in the generated at least one first image frame comprising at least one of a screen size, a screen resolution, an aspect ratio, a color profile, a brightness of screen, a wavelength, or other display-specific properties.

11. The method of claim 1, wherein the one or more user gesture features represent a characteristic of one or more motions and movements captured in the generated at least one first image frame comprising at least one of one or more hand gestures, one or more body postures, or other physical interactions with a visual content.

12. The method of claim 1, wherein the one or more ocular features represent a characteristic of one or more eye motions and eye movements of the user of the wearable device captured in the obtained at least one second image frame comprising at least one of pupil size information, spatial focus time matrix information, blinking rate information, eye-opening size pattern information, or eye tear condition information.

13. The method of claim 1,
wherein the type of the refractive error is determined based on the first feature and the second feature set, and
wherein the one or more content features and the one or more display features are dynamically adjusted based on the type of the refractive error and the determined refractive risk score.

14. A wearable device, comprising:
at least one camera,
memory storing one or more computer programs;
one or more processors communicatively coupled to the at least one camera and the memory,
wherein the one or more computer programs include computer-executable instructions that, when executed by the one or more processors individually or collectively, cause the wearable device to:
generate at least one first image frame for providing a virtual reality or an augmented reality through the wearable device;
obtain, through the at least one camera, at least one second image frame of an ocular of the user;
identify at least one first feature from the at least one first image frame, wherein the at least one first feature comprises at least one of one or more content features or one or more display features, and one or more user gesture features;
identify at least one second feature from the at least one second image frame, wherein the at least one second feature comprises one or more ocular features of the user;
based on the at least one first feature and the at least one second feature, determine a type of a refractive error of the user while the virtual reality or the augmented reality is provided;
adjust, based on the type of the refractive error, at least one of the identified content features or the identified display features, and
based on the at least one of the adjusted content features or the adjusted display features, providing the virtual reality or the augmented reality.

15. The wearable device of claim 14, wherein the one or more computer programs further include computer-executable instructions that, when executed by the one or more processors individually or collectively, cause the wearable device to:
determine a refractive risk score, wherein the refractive risk score is determined based on the first feature, the second feature, and the type of the refractive error, and
adjust the one or more content features and the one or more display features, in order to mitigate the determined refractive risk score, for dynamically correcting the refractive error of the user.

16. The wearable device of claim 14, wherein to determine the refractive error, the one or more computer programs further include computer-executable instructions that, when executed by the one or more processors individually or collectively, cause the wearable device to:
determine, based on the first feature and the second feature, a temporal feature vector for the generated at least one first image frame and the obtained at least one second image frame by utilizing a pre-trained autoregressive neural network;
determine, based on the one or more ocular features, a three-dimensional (3D) feature vector for the obtained at least one second image frame by utilizing a pre-trained multi-layer perceptron (MLP) neural network;
combine the determined temporal feature vector and the determined 3D feature vector into a single feature input vector, and
feed the single feature input vector into a pre-trained MLP classifier to determine the type of the refractive error based on a predefined threshold value.

17. The wearable device of claim 16,
wherein the pre-trained autoregressive neural network captures one or more temporal dynamics and dependencies present in a sequence of image frames, allowing for the extraction of a robust temporal representation,
wherein the pre-trained MLP neural network captures one or more intricate spatial and structural characteristics of an ocular region, enabling a robust representation of the one or more extracted ocular features,
wherein the 3D feature vector provides a compact and informative encoding of ocular-specific information present in the obtained at least one second image frame, and
wherein the type of the refractive error comprises at least one of myopia, hyperopia, astigmatism, and normal refractive status.

18. The wearable device of claim 15, wherein to determine the refractive risk score, the one or more computer programs further include computer-executable instructions that, when executed by the one or more processors individually or collectively, cause the system to:
obtain the type of the refractive error for a predefined set of consecutive image frames from the generated at least one first image frame and the obtained at least one second image frame;
determine, upon obtaining the type of the refractive error for the predefined set of consecutive image fames, a mean distribution of refractive errors by averaging distributions of the determined refractive error values obtained for the predefined set of consecutive image frames;
determine a mean refractive error by selecting a refractive error class with maximum probability in the determined mean distribution of refractive errors, and feed the determined mean distribution of refractive errors and the mean refractive error as input to a pre-trained long short-term memory (LSTM) neural network module to determine the refractive risk score.

19. The wearable device of claim 18, wherein the one or more computer programs further include computer-executable instructions that, when executed by the one or more processors individually or collectively, cause the system to:
store the determined refractive risk score in the wearable device, and generate a risk profile of the user based on the stored refractive risk score.

20. One or more non-transitory computer-readable storage media storing one or more computer programs including computer-executable instructions that, when executed by one or more processors of a wearable device individually or collectively, cause the wearable device to perform operations, the operations comprising:
generating at least one first image frame for providing a virtual reality or an augmented reality through the wearable device;
obtaining, through at least one camera of the wearable device, at least one second image frame of an ocular of the user;
identifying at least one first feature from the at least one first image frame, wherein the at least one first feature comprises at least one of one or more content features or one or more display features, and one or more user gesture features;
identifying at least one second feature from the at least one second image frame, wherein the at least one second feature comprises one or more ocular features of the user;
based on the at least one first feature and the at least one second feature, determining a type of a refractive error of the user while the virtual reality or the augmented reality is provided;
adjusting, based on the type of the refractive error, at least one of the identified content features or the identified display features, and
based on the at least one of the adjusted content features or the adjusted display features, providing the virtual reality or the augmented reality.
